(19) United States
(12) Patent Application Publication
(10) Pub. No.: US 2025/0033015 A1
(43) Pub. Date: Jan. 30, 2025

(54) POLYDISULFIDE CORE-SHELL MICROCAPSULES

(71) Applicant: Firmenich SA, Satigny (CH)

(72) Inventors: Ang LI, Plainsboro, NJ (US); Huda JERRI, Plainsboro, NJ (US); Christopher HANSEN, Plainsboro, NJ (US)

(21) Appl. No.: 18/717,271

(22) PCT Filed: Dec. 14, 2022
(86) PCT No.: PCT/EP22/85895

§ 371 Date: Jun. 6, 2024

Related U.S. Application Data
(60) Provisional application No. 63/290,779, filed on Dec. 17, 2021.

(30) Foreign Application Priority Data
Jan. 11, 2022 (EP) 22150844.3

(51) Int. Cl. B01J 13/18; A61K 8/11; A61Q 5/02; A61Q 5/12; A61Q 13/00; A61Q 15/00; A61Q 19/10; B01J 13/20; C11D 3/37; C11D 3/50

(52) U.S. Cl. CPC B01J 13/185; A61K 8/11; A61Q 5/02; A61Q 5/12; A61Q 13/00; A61Q 15/00; A61Q 19/10; B01J 13/206; C11D 3/378; C11D 3/505; A61K 2800/52; A61K 2800/54; A61K 2800/56; A61K 2800/652; A61K 2800/95; C11D 2111/12

(57) ABSTRACT

Disclosed herein is a process for the preparation of polydisulfide core-shell microcapsules. The polydisulfide core-shell microcapsules include an oil-based core including a hydrophobic material and a polymeric shell including polydisulfide bonds. Polydisulfide core-shell microcapsules are also disclosed herein. A perfuming composition and a consumer product including polydisulfide core-shell microcapsules are also disclosed herein.

### TECHNICAL FIELD

The present invention relates to a new process for the preparation of polydisulfide core-shell microcapsules. The polydisulfide core-shell microcapsule comprises an oil-based core comprising a hydrophobic material and a polymeric shell comprising polydisulfide bonds. Polydisulfide core-shell microcapsules are also an object of this invention. A perfuming composition and a consumer product comprising polydisulfide core-shell microcapsules are also an object of this invention.

### BACKGROUND OF THE INVENTION

One of the problems faced by the perfumery industry lies in the relatively rapid loss of olfactive benefit provided by odoriferous compounds due to their high volatility, particularly that of “top-notes”. In order to tailor the release rates of volatiles, delivery system such as microcapsules containing active ingredients, for example a perfume, are needed to protect and later release the core payload when triggered. A key requirement from the industry regarding these systems is to survive suspension in challenging bases without physically dissociating or degrading. This is referred to as chemical stability of a delivery system. For instance, fragrance personal and household cleansers containing high levels of aggressive surfactant detergents are very challenging for the stability of delivery systems, such as microcapsules. High levels of surfactants also increase the speed of diffusion of actives out of the delivery system, such as a microcapsule. This leads to leakage of the actives during storage and a reduced impact when the microcapsules are triggered to release.

In addition to the performance in terms of stability and olfactive performance, the consumer demand for eco-friendly delivery systems is more and more important and is driving the development of new delivery systems. There is still a need in the industry for improving the biodegradability and at the same time provide a certain degree of chemical stability for a delivery system and reducing the leakage of a perfume from the delivery system and preferably providing an improved impact of the perfume upon release to the consumer.

There is therefore still a need to provide new microcapsules using more eco-friendly materials, while not compromising on the performance of the microcapsules, in particular in terms of stability in a challenging medium such as a consumer product base, as well as in delivering a good performance in terms of active ingredient delivery, e.g. olfactive performance in the case of perfuming ingredients.

The present invention satisfies these and other needs of the industry.

### DETAILED DESCRIPTION OF THE INVENTION

Unless stated otherwise, percentages (%) are meant to designate percent by weight (wt. %) of a composition.

Room temperature is herein understood as a temperature between 20° C. and 25° C. or between 293 K and 298 K.

By “hydrophobic material”, it is meant a material which forms a two-phase dispersion when mixed with water. According to the invention, the hydrophobic material can be “inert” material like solvents or active ingredients. According to an embodiment, the hydrophobic material is a hydrophobic active ingredient.

By “active ingredient”, it is meant a single compound or a combination of ingredients.

By “perfume oil”, it is meant a single perfuming or a mixture of several perfuming compounds.

By “consumer product” or “end-product” it is meant a manufactured product ready to be distributed, sold and used by a consumer.

A “core-shell microcapsule”, or the similar, in the present invention comprises a core based on a hydrophobic material, typically a perfume oil, and a polymeric shell surrounding the oil core.

Core-shell microcapsules have a particle size distribution in the micron range (e.g. a mean diameter) comprised between about 1 and 3000 microns, preferably comprised between 1 and 1000 microns, more preferably between 1 and 500 microns, and even more preferably between 5 and 50 microns.

By “particle size” it is meant an average diameter of particles based on size distribution measured by dynamic light scattering (DLS) using Zetasizer Nano ZS equipment from Malvern Instruments Ltd., UK when particles are dispersed into a water phase.

By “microcapsules size” it is meant the volume mean diameter of the relevant capsules, capsules suspension as obtained by laser light scattering of a diluted sample in a Malvern Mastersizer 3000.

By “core-shell microcapsule slurry”, it is meant core-shell microcapsule(s) that is (are) dispersed in a liquid. According to an embodiment, the slurry is an aqueous slurry, i.e the core-shell microcapsule(s) is (are) dispersed in an aqueous phase.

By “polydisulfide core-shell microcapsule slurry”, it is meant polydisulfide core-shell microcapsule(s) that is (are) dispersed in a liquid. According to an embodiment, the slurry is an aqueous slurry, i.e the core-shell microcapsule(s) is (are) dispersed in an aqueous phase.

By “polyfunctional monomer”, it is meant a molecule that, as unit, reacts or binds chemically to form a polymer or a supramolecular polymer. The polyfunctional monomer of the invention has at least two functional groups that are capable to react with or bind to functional groups of another component and/or are capable to polymerize to form a polymeric shell.

### Process for the Preparation of a Polydisulfide Core-Shell Microcapsule Slurry

The present invention relates to a process for the preparation of a polydisulfide core-shell microcapsule slurry, comprising the steps of
a) dissolving at least one polythiol monomer in a hydrophobic material, preferably a perfume oil, to form an oil phase; and
b) dispersing the oil phase obtained in step a) into an aqueous phase to form an oil-in-water emulsion; and
c) applying sufficient conditions to induce thiol oxidation at the oil-water interface to form a polydisulfide core-shell microcapsule slurry,
wherein an oxidation catalyst is added in the oil phase in step a), and/or an oxidation catalyst is added in the oil-in-water emulsion obtained after step b), and
wherein a stabilizer is added in the oil phase in step a) and/or in the aqueous phase in step b), and
wherein an oxidant is added in the aqueous phase in step b) and/or in the oil-in-water emulsion obtained after step b).

According to an embodiment, the process comprises the steps of
a) dissolving at least one polythiol monomer in a hydrophobic material, preferably a perfume oil, to form an oil phase; and
b) dispersing the oil phase obtained in step a) into an aqueous phase to form an oil-in-water emulsion; and
c) adding an oxidant in the oil-in-water emulsion under stirring to induce thiol oxidation at the oil-water interface to form a polydisulfide core-shell microcapsule slurry,
wherein an oxidation catalyst is added in the oil phase in step a), and/or an oxidation catalyst is added in the oil-in-water emulsion obtained after step b), and
wherein a stabilizer is added in the oil phase in step a) and/or in the aqueous phase in step b).

### Polythiol Monomer

According to the present invention, an oil phase is formed by dissolving at least one polythiol monomer in a hydrophobic material, preferably a perfume oil.

According to an embodiment, polythiol monomers are dissolved in a hydrophobic material.

A polythiol monomer is herein understood to be an organic molecule comprising two or more functional groups that are thiol groups. The term thiol group is herein understood to be a —SH functional group. A person skilled in the art is aware of the definition of a thiol group.

According to one embodiment, the polythiol monomer is chosen in the group consisting of difunctional thiol monomers, trifunctional thiol monomers, tetrafunctional thiol monomers, pentafunctional thiol monomers, hexafunctional thiol monomers, heptafunctional thiol monomers, octafunctional thimonomers, thiolated polysaccharides, thiolated chitosan, thiolated proteins, thiolated pectin, thiolated polyols, hydrolysed/reduced keratin, proteins with two or more thiol groups and mixtures thereof.

According to one embodiment, the polythiol monomer is a difunctional thiol monomer, such as 1,2-ethanedithiol 1,3-propanedithiol, 1,4-butanedithiol, 1,5-pentanedithiol, 1,6-hexanedithiol, 1,8-octanedithiol, 1,9-nonanedithiol, dithioerythritol, 1,4-butanediol bis(thioglycolate), 4,4′-biphenyldithiol, ethylene bis(thioglycolate), ethylene glycol bis(3-mercaptopropionate), 1,10-decanedithiol, 1,11-undecanedithiol, 1,16-hexadecanedithiol, 1,4-benzenedithiol, 3,7-dithia-1,9-nonanedithiol, 4,4′-thiobisbenzenethiol, hexa (ethylene glycol) dithiol, bis(2-mercaptoethyl) sulfide, 3,6-dioxa-1,8-octanedithiol, 1,5-dimercaptonaphthalene, 1,3-benzenedithiol, 2,3-butanedithiol, dithiothreitol, 2,3-dimercapto-1-propanol 2,2′-(ethylenedioxy) diethanethiol, hexa (ethylene glycol) dithiol, tetra(ethylene glycol) dithiol, polyethylene glycol dithiol, preferably ethylene glycol bis(3-mercaptopropionate), or mixtures thereof.

Polyethylene glycol dithiol can have a molecular weight (Mₙ) of 1000 g/mol to 8000 g/mol, preferably of 1500 g/mol to 3400 g/mol.

When a difunctional thiol monomer is used, it can be used in combination with at least one polythiol monomer having more than two —SH functional group, such as for example trifunctional thiol monomers, tetrafunctional thiol monomers, pentafunctional thiol monomers, hexafunctional thiol monomers, heptafunctional thiol monomers or octafunctional thimonomers.

According to one embodiment, the polythiol monomer is a trifunctional thiol monomer, preferably trimethylolpropane tris(3-mercaptopropionate) or tris[2-(3-mercaptopropionyloxy)ethyl] isocyanurate, preferably tris[2-(3-mercaptopropionyloxy)ethyl] isocyanurate.

According to one embodiment, the polythiol monomer is a tetrafunctional thiol monomer, preferably pentaerythritol tetrakis(3-mercaptopropionate) or 4arm-PEG-SH. 4arm-PEG-SH is herein understood to be a component of chemical formula C(CH₂O(CH₂CH₂O)ₙCH₂CH₂SH)₄ and wherein preferably n is an integer so that the compound has a molecular weight (Mₙ) of 5000 g/mol to 20000 g/mol. 4arm-PEG-SH can have a molecular weight (Mₙ) of 5000 g/mol to 20000 g/mol, preferably of 8000 g/mol to 15000 g/mol.

According to one embodiment, the polythiol monomer is a hexafunctional thiol monomer, preferably dipentaerythritol hexakis (3-mercaptopropionate).

According to one embodiment, the polythiol monomers is an octafunctional thiol monomer, preferably 8arm-PEG-SH. 8arm-PEGSH is herein understood to be a component of chemical formula R(O(CH₂CH₂O)nCH₂CH₂SH)₈ wherein R is a tripentaerythritol or hexaglycerol core structure and wherein preferably n is an integer so that the compound has a molecular weight (Mₙ) of 10000 g/mol to 20000 g/mol. 8arm-PEG-SH can have a molecular weight (Mₙ) of 10000 g/mol to 20000 g/mol, preferably of 13000 g/mol to 17000 g/mol.

According to one embodiment, the polythiol monomer is present in an amount of 0.1 wt. % to 30 wt. %, preferably 0.5 wt. % to 25 wt. %, more preferably 1.0 wt. % to 15 wt. %, based on the total weight of the oil-in water emulsion obtained in step b).

According to an embodiment, the polythiol monomer is present in an amount of 0.1 wt. % to 50 wt. %, preferably 0.5 wt. % to 35 wt. % and more preferably 1 wt. % to 25 wt. % based on the total amount of the oil phase.

According to one embodiment, at least one further polyfunctional monomer is added to the process, wherein the at least one further polyfunctional monomer is no polythiol monomer. According to a preferred embodiment, the at least one further polyfunctional monomer is added to the oil phase in step a).

According to an embodiment, the at least one further polyfunctional monomer is chosen in the group consisting of polyisocyanate, polyanhydride such as poly maleic anhydride, poly acyl chloride, polyepoxide, polyaldehyde, polyalkoxysilane, alkene, alkyne, or acrylate monomers such as poly(meth)acrylate monomers, whereas poly(meth)acrylate comprises polyacrylates, polymethacrylates and mixture of polyacrylate and polymethacrylate, and mixtures thereof.

According to a particular embodiment, the at least one other polyfunctional monomer added in step a) is at least one polyisocyanate having at least two isocyanate functional groups.

Suitable polyisocyanates used according to this embodiment include aromatic polyisocyanate, aliphatic polyisocyanate and mixtures thereof. Said polyisocyanate comprises at least 2, preferably at least 3 but may comprise up to 6, or even only 4, isocyanate functional groups. According to a particular embodiment, a triisocyanate is used.

According to one embodiment, said polyisocyanate is an aromatic polyisocyanate.

The term “aromatic polyisocyanate” is meant here as encompassing any polyisocyanate comprising an aromatic moiety. Preferably, it comprises a phenyl, a toluyl, a xylyl, a naphthyl or a diphenyl moiety, more preferably a toluyl or a xylyl moiety. Preferred aromatic polyisocyanates are biurets, polyisocyanurates and trimethylol propane adducts of diisocyanates, more preferably comprising one of the above-cited specific aromatic moieties. More preferably, the aromatic polyisocyanate is a polyisocyanurate of toluene diisocyanate (commercially available from Bayer under the tradename Desmodur® RC), a trimethylol propane-adduct of toluene diisocyanate (commercially available from Bayer under the tradename Desmodur® L75), a trimethylol propane-adduct of xylylene diisocyanate (commercially available from Mitsui Chemicals under the tradename Takenate® D-110N). In a most preferred embodiment, the aromatic polyisocyanate is a trimethylol propane-adduct of xylylene diisocyanate.

According to another embodiment, said polyisocyanate is an aliphatic polyisocyanate. The term “aliphatic polyisocyanate” is defined as a polyisocyanate which does not comprise any aromatic moiety. Preferred aliphatic polyisocyanates are a trimer of hexamethylene diisocyanate, a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100), among which a biuret of hexamethylene diisocyanate is even more preferred.

According to another embodiment, the at least one polyisocyanate is in the form of a mixture of at least one aliphatic polyisocyanate and of at least one aromatic polyisocyanate, both comprising at least two or three isocyanate functional groups, such as a mixture of a biuret of hexamethylene diisocyanate with a trimethylol propane-adduct of xylylene diisocyanate, a mixture of a biuret of hexamethylene diisocyanate with a polyisocyanurate of toluene diisocyanate and a mixture of a biuret of hexamethylene diisocyanate with a trimethylol propane-adduct of toluene diisocyanate. Most preferably, it is a mixture of a biuret of hexamethylene diisocyanate with a trimethylol propane-adduct of xylylene diisocyanate. Preferably, when used as a mixture the molar ratio between the aliphatic polyisocyanate and the aromatic polyisocyanate is ranging from 80:20 to 10:90.

According to an embodiment, the at least one polyfunctional monomer used in the process of this embodiment is present in amounts representing from 0.1 to 35%, preferably from 0.5 to 10% and more preferably from 0.8 to 6%, and even more preferably between 1 and 5% by weight, based on the total amount of the oil phase.

### Hydrophobic Material

According to an embodiment, the core is an oil-based core.

The hydrophobic material according to the invention can be “inert” material like solvents or active ingredients.

When the hydrophobic material is an active ingredient, it is preferably chosen from the group consisting of flavor, flavor ingredients, perfume, perfume ingredients, nutraceuticals, cosmetics, pest control agents, biocide actives, malodour counteracting ingredient, bactericide ingredient, fungicide ingredient, pharmaceutical or agrochemical ingredient, a sanitizing ingredient, an insect repellent or attractant, and mixtures thereof.

According to a particular embodiment, the hydrophobic material comprises a phase change material (PCM).

According to a particular embodiment, the hydrophobic material comprises a mixture of a perfume with another ingredient selected from the group consisting of nutraceuticals, cosmetics, pest control agents and biocide actives.

According to a particular embodiment, the hydrophobic material comprises a mixture of biocide actives with another ingredient selected from the group consisting of perfumes, nutraceuticals, cosmetics or pest control agents.

According to a particular embodiment, the hydrophobic material comprises a mixture of pest control agents with another ingredient selected from the group consisting of perfumes, nutraceuticals, cosmetics, biocide actives.

According to a particular embodiment, the hydrophobic material comprises a perfume.

According to a particular embodiment, the hydrophobic material consists of a perfume.

According to a particular embodiment, the hydrophobic material consists of biocide actives.

According to a particular embodiment, the hydrophobic material consists of pest control agents.

By “perfume” (or also “perfume oil”) what is meant here is an ingredient or a composition that is a liquid at about 20° C. According to any one of the above embodiments said perfume oil can be a perfuming ingredient alone or a mixture of ingredients in the form of a perfuming composition. As a “perfuming ingredient” it is meant here a compound, which is used for the primary purpose of conferring or modulating an odor. In other words such an ingredient, to be considered as being a perfuming one, must be recognized by a person skilled in the art as being able to at least impart or modify in a positive or pleasant way the odor of a composition, and not just as having an odor. For the purpose of the present invention, perfume oil also includes a combination of perfuming ingredients with substances which together improve, enhance or modify the delivery of the perfuming ingredients, such as perfume precursors, modulators, emulsions or dispersions, as well as combinations which impart an additional benefit beyond that of modifying or imparting an odor, such as long-lastingness, blooming, malodor counteraction, antimicrobial effect, microbial stability and/or pest control.

The nature and type of the perfuming ingredients present in the oil phase do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, acetates, nitriles, terpenoids, nitrogenous or sulfurous heterocyclic compounds and essential oils (for example Thyme oil), and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery.

In particular one may cite perfuming ingredients which are commonly used in perfume formulations, such as:
Aldehydic ingredients: decanal, dodecanal, 2-methyl-undecanal, 10-undecenal, octanal, nonanal and/or nonenal;
Aromatic-herbal ingredients: eucalyptus oil, camphor, eucalyptol, 5-methyltricyclo[6.2.1.0^{2.7}]undecan-4-one, 1-methoxy-3-hexanethiol, 2-ethyl-4,4-dimethyl-1,3-oxathiane, 2,2,7/8,9/10-tetramethylspiro[5.5]undec-8-en-1-one, menthol and/or alpha-pinene;
Balsamic ingredients: coumarin, ethylvanillin and/or vanillin;
Citrus ingredients: dihydromyrcenol, citral, orange oil, linalyl acetate, citronellyl nitrile, orange terpenes, limonene, 1-p-menthen-8-yl acetate and/or 1,4(8)-p-menthadiene;
Floral ingredients: methyl dihydrojasmonate, linalool, citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4 (2H)-pyranol, beta ionone, methyl 2-(methylamino) benzoate, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1-[2,6,6-trimethyl-3-cyclohexen-1-yl]-2-buten-1-one, (2E)-1-(2,6,6-trimethyl-1-cyclohexen-1-yl)-2-buten-1-one, 3-(3,3/1, 1-dimethyl-5-indanyl) propanal, 2,5-dimethyl-2-indanmethanol, 2,6,6-trimethyl-3-cyclohexene-1-carboxylate, 3-(4,4-dimethyl-1-cyclohexen-1-yl) propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, propyl(S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionones isomers;
Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, diethyl 1,4-cyclohexanedicarboxylate, 3-methyl-2-hexen-1-yl acetate, 1-[3,3-dimethylcyclohexyl]ethyl [3-ethyl-2-oxiranyl]acetate and/or diethyl 1,4-cyclohexane dicarboxylate;
Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl(2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{(1S)-1-[(1R)-3,3-dimethylcyclohexyl]ethoxy}-2-oxoethyl propionate, 3-methyl-5-cyclopentadecen-1-one, 4,6,6,7,8,8-hexamethyl-1,3,4,6,7,8-hexahydrocyclopenta[g]isochromene, (1S,1′R)-2-[1-(3′,3′-dimethyl-1′-cyclohexyl)ethoxy]-2-methylpropyl propanoate, oxacyclohexadecan-2-one and/or (1S,1′R)-[1-(3′,3′-dimethyl-1′-cyclohexyl)ethoxycarbonyl]methyl propanoate;
Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2-ol, 3,4′-dimethylspiro[oxirane-2,9′-tricyclo[6.2.1.0^{2.7}]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, patchouli oil, terpenes fractions of patchouli oil, Clearwood®, (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl) ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal, 7-methyl-2H-1,5-benzodioxepin-3(4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonane and/or 3-(3-isopropyl-1-phenyl)butanal.

It is also understood that said ingredients may also be compounds known to release in a controlled manner various types of perfuming compounds also known as properfume or profragrance. Non-limiting examples of suitable properfumes may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, 3-(dodecylthio)-1-(2,6,6-trimethyl-3-cyclohexen-1-yl)-1-butanone, 2-(dodecylthio) octan-4-one, 2-phenylethyl oxo (phenyl)acetate, 3,7-dimethylocta-2,6-dien-1-yl oxo (phenyl)acetate, (Z)-hex-3-en-1-yl oxo (phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy) undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-4-((2-methoxy-2-phenylvinyl)oxy)benzaldehyde, 4-((2-(hexyloxy)-2-phenylvinyl)oxy)-3-methoxybenzaldehyde or a mixture thereof.

The perfuming ingredients may be dissolved in a solvent of current use in the perfume industry. The solvent is preferably not an alcohol. Examples of such solvents are diethyl phthalate, isopropyl myristate, Abalyn® (rosin resins, available from Eastman), benzyl benzoate, ethyl citrate, triethyl citrate, limonene or other terpenes, or isoparaffins. Preferably, the solvent is very hydrophobic and highly sterically hindered, like for example Abalyn® or benzyl benzoate. Preferably the perfume comprises less than 30% of solvent. More preferably the perfume comprises less than 20% and even more preferably less than 10% of solvent, all these percentages being defined by weight relative to the total weight of the perfume. Most preferably, the perfume is essentially free of solvent.

Preferred perfuming ingredients are those having a high steric hindrance (bulky materials) and in particular those from one of the following groups:
Group 1: perfuming ingredients comprising a cyclohexane, cyclohexene, cyclohexanone or cyclohexenone ring substituted with at least one linear or branched C₁ to C₄ alkyl or alkenyl substituent;
Group 2: perfuming ingredients comprising a cyclopentane, cyclopentene, cyclopentanone or cyclopentenone ring substituted with at least one linear or branched C₄ to C₈ alkyl or alkenyl substituent;
Group 3: perfuming ingredients comprising a phenyl ring or perfuming ingredients comprising a cyclohexane, cyclohexene, cyclohexanone or cyclohexenone ring substituted with at least one linear or branched C₅ to C₈ alkyl or alkenyl substituent or with at least one phenyl substituent and optionally one or more linear or branched C₁ to C₃ alkyl or alkenyl substituents;
Group 4: perfuming ingredients comprising at least two fused or linked C₅ and/or C₆ rings;
Group 5: perfuming ingredients comprising a camphor-like ring structure;
Group 6: perfuming ingredients comprising at least one C₇ to C₂₀ ring structure;
Group 7: perfuming ingredients having a log P value above 3.5 and comprising at least one tert-butyl or at least one trichloromethyl substitutent;

Examples of ingredients from each of these groups are:
Group 1: 2,4-dimethyl-3-cyclohexene-1-carbaldehyde (origin: Firmenich SA, Geneva, Switzerland), isocyclocitral, menthone, isomenthone, methyl 2,2-dimethyl-6-methylene-1-cyclohexanecarboxylate (origin: Firmenich SA, Geneva, Switzerland), nerone, terpineol, dihydroterpineol, terpenyl acetate, dihydroterpenyl acetate, dipentene, eucalyptol, hexylate, rose oxide, (S)-1,8-p-menthadiene-7-ol (origin: Firmenich SA, Geneva, Switzerland), 1-p-menthene-4-ol, (1RS,3RS,4SR)-3-p-mentanyl acetate, (1R,2S,4R)-4,6,6-trimethyl-bicyclo[3,1,1]heptan-2-ol, tetrahydro-4-methyl-2-phenyl-2H-pyran (origin: Firmenich SA, Geneva, Switzerland), cyclohexyl acetate, cyclanol acetate, 1,4-cyclohexane diethyldicarboxylate (origin: Firmenich SA, Geneva, Switzerland), (3RS,3aRS,6SR,7ASR)-perhydro-3,6-dimethyl-benzo[B]furan-2-one (origin: Firmenich SA, Geneva, Switzerland), ((6R)-perhydro-3,6-dimethyl-benzo[B]furan-2-one (origin: Firmenich SA, Geneva, Switzerland), 2,4,6-trimethyl-4-phenyl-1,3-dioxane, 2,4,6-trimethyl-3-cyclohexene-1-carbaldehyde;
Group 2: (E)-3-methyl-5-(2,2,3-trimethyl-3-cyclopenten-1-yl)-4-penten-2-ol (origin: Givaudan SA, Vernier, Switzerland), (1′R,E)-2-ethyl-4-(2′,2′,3′-trimethyl-3′-cyclopenten-1′-yl)-2-buten-1-ol (origin: Firmenich SA, Geneva, Switzerland), (1′R,E)-3,3-dimethyl-5-(2′,2′,3′-trimethyl-3′-cyclopenten-1′-yl)-4-penten-2-ol (origin: Firmenich SA, Geneva, Switzerland), 2-heptylcyclopentanone, methyl-cis-3-oxo-2-pentyl-1-cyclopentane acetate (origin: Firmenich SA, Geneva, Switzerland), 2,2,5-trimethyl-5-pentyl-1-cyclopentanone (origin: Firmenich SA, Geneva, Switzerland), 3,3-dimethyl-5-(2,2,3-trimethyl-3-cyclopenten-1-yl)-4-penten-2-ol (origin: Firmenich SA, Geneva, Switzerland), 3-methyl-5-(2,2,3-trimethyl-3-cyclopenten-1-yl)-2-pentanol (origin, Givaudan SA, Vernier, Switzerland);
Group 3: damascones, 1-(5,5-dimethyl-1-cyclohexen-1-yl)-4-penten-1-one (origin: Firmenich SA, Geneva, Switzerland), (1′R)-2-[2-(4′-methyl-3′-cyclohexen-1′-yl)propyl]cyclopentanone, alpha-ionone, beta-ionone, damascenone, mixture of 1-(5,5-dimethyl-1-cyclohexen-1-yl)-4-penten-1-one and 1-(3,3-dimethyl-1-cyclohexen-1-yl)-4-penten-1-one (origin: Firmenich SA, Geneva, Switzerland), 1-(2,6,6-trimethyl-1-cyclohexen-1-yl)-2-buten-1-one (origin: Firmenich SA, Geneva, Switzerland), (1S,1′R)-[1-(3′,3′-Dimethyl-1′-cyclohexyl)ethoxycarbonyl]methyl propanoate (origin: Firmenich SA, Geneva, Switzerland), 2-tert-butyl-1-cyclohexyl acetate (origin: International Flavors and Fragrances, USA), 1-(2,2,3,6-tetramethyl-cyclohexyl)-3-hexanol (origin: Firmenich SA, Geneva, Switzerland), trans-1-(2,2,6-trimethyl-1-cyclohexyl)-3-hexanol (origin: Firmenich SA, Geneva, Switzerland), (E)-3-methyl-4-(2,6,6-trimethyl-2-cyclohexen-1-yl)-3-buten-2-one, terpenyl isobutyrate, 4-(1,1-dimethylethyl)-1-cyclohexyl acetate (origin: Firmenich SA, Geneva, Switzerland), 8-methoxy-1-p-menthene, (1S,1′R)-2-[1-(3′,3′-dimethyl-1′-cyclohexyl)ethoxy]-2-methylpropyl propanoate (origin: Firmenich SA, Geneva, Switzerland), para tert-butylcyclohexanone, menthenethiol, 1-methyl-4-(4-methyl-3-pentenyl)-3-cyclohexene-1-carbaldehyde, allyl cyclohexylpropionate, cyclohexyl salicylate, 2-methoxy-4-methylphenyl methyl carbonate, ethyl 2-methoxy-4-methylphenyl carbonate, 4-ethyl-2-methoxyphenyl methyl carbonate;
Group 4: Methyl cedryl ketone (origin: International Flavors and Fragrances, USA), a mixture of (1RS,2SR,6RS,7RS,8SR)-tricyclo[5.2.1.0^{2.6}]dec-3-en-8-yl 2-methylpropanoate and (1RS,2SR,6RS,7RS,8SR)-tricyclo[5.2.1.0^{2.6}]dec-4-en-8-yl 2-methylpropanoate, vetyverol, vetyverone, 1-(octahydro-2,3,8,8-tetramethyl-2-naphtalenyl)-1-ethanone (origin: International Flavors and Fragrances, USA), (5RS,9RS,10SR)-2,6,9,10-tetramethyl-1-oxaspiro[4.5]deca-3,6-diene and the (5RS,9SR, 10RS) isomer, 6-ethyl-2,10,10-trimethyl-1-oxaspiro[4.5]deca-3,6-diene, 1,2,3,5,6,7-hexahydro-1,1,2,3,3-pentamethyl-4-indenone (origin: International Flavors and Fragrances, USA), a mixture of 3-(3,3-dimethyl-5-indanyl) propanal and 3-(1,1-dimethyl-5-indanyl) propanal (origin: Firmenich SA, Geneva, Switzerland), 3′,4-dimethyl-tricyclo[6.2.1.0(2,7)]undec-4-ene-9-spiro-2′-oxirane (origin: Firmenich SA, Geneva, Switzerland), 9/10-ethyldiene-3-oxatricyclo[6.2.1.0(2,7)]undecane, (perhydro-5,5,8A-trimethyl-2-naphthalenyl acetate (origin: Firmenich SA, Geneva, Switzerland), octalynol, (dodecahydro-3a,6,6,9a-tetramethyl-naphtho [2,1-b]furan, origin: Firmenich SA, Geneva, Switzerland), tricyclo[5.2.1.0(2,6)]dec-3-en-8-yl acetate and tricyclo[5.2.1.0(2,6)]dec-4-en-8-yl acetate as well as tricyclo[5.2.1.0(2,6)]dec-3-en-8-yl propanoate and tricyclo[5.2.1.0(2,6)]dec-4-en-8-yl propanoate, (+)-(1S,2S,3S)-2,6,6-trimethyl-bicyclo[3.1.1]heptane-3-spiro-2′-cyclohexen-4′-one;
Group 5: camphor, borneol, isobornyl acetate, 8-isopropyl-6-methyl-bicyclo[2.2.2]oct-5-ene-2-carbaldehyde, pinene, camphene, 8-methoxycedrane, (8-methoxy-2,6,6,8-tetramethyl-tricyclo[5.3.1.0(1,5)]undecane (origin: Firmenich SA, Geneva, Switzerland), cedrene, cedrenol, cedrol, mixture of 9-ethylidene-3-oxatricyclo[6.2.1.0(2,7)]undecan-4-one and 10-ethylidene-3-oxatricyclo[6.2.1.0^{2.7}]undecan-4-one (origin: Firmenich SA, Geneva, Switzerland), 3-methoxy-7,7-dimethyl-10-methylene-bicyclo[4.3.1]decane (origin: Firmenich SA, Geneva, Switzerland);
Group 6: (trimethyl-13-oxabicyclo-[10.1.0]-trideca-4,8-diene (origin: Firmenich SA, Geneva, Switzerland), 9-hexadecen-16-olide (origin: Firmenich SA, Geneva, Switzerland), pentadecenolide (origin: Firmenich SA, Geneva, Switzerland), 3-methyl-(4/5)-cyclopentadecenone, (origin: Firmenich SA, Geneva, Switzerland), 3-methylcyclopentadecanone (origin: Firmenich SA, Geneva, Switzerland), pentadecanolide (origin: Firmenich SA, Geneva, Switzerland), cyclopentadecanone (origin: Firmenich SA, Geneva, Switzerland), 1-ethoxyethoxy)cyclododecane (origin: Firmenich SA, Geneva, Switzerland), 1,4-dioxacycloheptadecane-5,17-dione, 4,8-cyclododecadien-1-one;
Group 7: (+−)-2-methyl-3-[4-(2-methyl-2-propanyl)phenyl]propanal (origin: Givaudan SA, Vernier, Switzerland), 2,2,2-trichloro-1-phenylethyl acetate.

Preferably, the perfume comprises at least 30%, preferably at least 50%, more preferably at least 60% of ingredients selected from Groups 1 to 7, as defined above. More preferably said perfume comprises at least 30%, preferably at least 50% of ingredients from Groups 3 to 7, as defined above. Most preferably said perfume comprises at least 30%, preferably at least 50% of ingredients from Groups 3, 4, 6 or 7, as defined above.

According to another preferred embodiment, the perfume comprises at least 30%, preferably at least 50%, more preferably at least 60% of ingredients having a log P above 3, preferably above 3.5 and even more preferably above 3.75.

According to a particular embodiment, the perfume used in the invention contains less than 10% of its own weight of primary alcohols, less than 15% of its own weight of secondary alcohols and less than 20% of its own weight of tertiary alcohols. Advantageously, the perfume used in the invention does not contain any primary alcohols and contains less than 15% of secondary and tertiary alcohols.

According to an embodiment, the oil phase (or the oil-based core) comprises:
25-100 wt %, preferably 25-98%, of a perfume oil comprising at least 15 wt % of high impact perfume raw materials having a Log T<−4, and
0-75 wt %, preferably 2-75% of a density balancing material having a density greater than 1.07 g/cm³.

“High impact perfume raw materials” should be understood as perfume raw materials having a Log T<−4. The odor threshold concentration of a chemical compound is determined in part by its shape, polarity, partial charges and molecular mass. For convenience, the odor threshold concentration is presented as the common logarithm of the threshold concentration, i.e., Log [Threshold] (“Log T”).

A “density balancing material” should be understood as a material having a density greater than 1.07 g/cm³ and having preferably low or no odor.

The odor threshold concentration of a perfuming compound is determined by using a gas chromatograph (“GC”). Specifically, the gas chromatograph is calibrated to determine the exact volume of the perfume oil ingredient injected by the syringe, the precise split ratio, and the hydrocarbon response using a hydrocarbon standard of known concentration and chain-length distribution. The air flow rate is accurately measured and, assuming the duration of a human inhalation to last 12 seconds, the sampled volume is calculated. Since the precise concentration at the detector at any point in time is known, the mass per volume inhaled is known and hence the concentration of the perfuming compound. To determine the threshold concentration, solutions are delivered to the sniff port at the back-calculated concentration. A panelist sniffs the GC effluent and identifies the retention time when odor is noticed. The average across all panelists determines the odor threshold concentration of the perfuming compound. The determination of odor threshold is described in more detail in C. Vuilleumier et al., Multidimensional Visualization of Physical and Perceptual Data Leading to a Creative Approach in Fragrance Development, Perfume & Flavorist, Vol. 33, September, 2008, pages 54-61.

The nature of high impact perfume raw materials having a Log T<−4 and density balancing material having a density greater than 1.07 g/cm3 are described in WO2018115250, the content of which are included by reference.

According to an embodiment, the high impact perfume raw materials having a Log T<−4 are selected from the group consisting of (+−)-1-methoxy-3-hexanethiol, 4-(4-hydroxy-1-phenyl)-2-butanone, 2-methoxy-4-(1-propenyl)-1-phenyl acetate, pyrazobutyle, 3-propylphenol, 1-(3-methyl-1-benzofuran-2-yl) ethanone, 2-(3-phenylpropyl)pyridine, 1-(3,3/5,5-dimethyl-1-cyclohexen-1-yl)-4-penten-1-one, 1-(5,5-dimethyl-1-cyclohexen-1-yl)-4-penten-1-one, a mixture comprising (3RS,3aRS,6SR,7ASR)-perhydro-3,6-dimethyl-benzo[b]furan-2-one and (3SR,3aRS,6SR,7ASR)-perhydro-3,6-dimethyl-benzo[b]furan-2-one, (+−)-1-(5-ethyl-5-methyl-1-cyclohexen-1-yl)-4-penten-1-one, (1'S,3′R)-1-methyl-2-[(1′,2′,2′-trimethylbicyclo[3.1.0]hex-3′-yl)methyl]cyclopropyl}methanol, (+−)-3-mercaptohexyl acetate, (2E)-1-(2,6,6-trimethyl-1,3-cyclohexadien-1-yl)-2-buten-1-one, H-methyl-2h-1,5-benzodioxepin-3 (4H)-one, (2E,6Z)-2,6-nonadien-1-ol, (4Z)-4-dodecenal, (+−)-4-hydroxy-2,5-dimethyl-3 (2H)-furanone, methyl 2,4-dihydroxy-3,6-dimethylbenzoate, 3-methylindole, (+−)-perhydro-4alpha,8abeta-dimethyl-4a-naphthalenol, patchoulol, 2-methoxy-4-(1-propenyl) phenol, mixture comprising (+−)-5,6-dihydro-4-methyl-2-phenyl-2H-pyran and tetrahydro-4-methylene-2-phenyl-2H-pyran, mixture comprising 4-methylene-2-phenyltetrahydro-2H-pyran and (+−)-4-methyl-2-phenyl-3,6-dihydro-2H-pyran, 4-hydroxy-3-methoxybenzaldehyde, nonylenic aldehyde, 2-methoxy-4-propylphenol, 3-methyl-5-phenyl-2-pentenenitrile, 1-(spiro[4.5]dec-6/7-en-7-yl)-4-penten-1-one, 2-methoxynaphthalene, (−)-(3aR,5AS,9AS,9BR)-3a,6,6,9a-tetramethyldodecahydronaphtho [2,1-b]furan, 5-nonanolide, (3aR,5AS,9AS,9BR)-3a,6,6,9a-tetramethyldodecahydronaphtho [2,1-b]furan, 7-isopropyl-2H,4H-1,5-benzodioxepin-3-one, coumarin, 4-methylphenyl isobutyrate, (2E)-1-(2,6,6-trimethyl-1,3-cyclohexadien-1-yl)-2-buten-1-one, beta,2,2,3-tetramethyl-delta-methylene-3-cyclopentene-1-butanol, delta damascone ((2E)-1-[(1RS,2SR)-2,6,6-trimethyl-3-cyclohexen-1-yl]-2-buten-1-one), (+−)-3,6-dihydro-4,6-dimethyl-2-phenyl-2h-pyran, anisaldehyde, paracresol, 3-ethoxy-4-hydroxybenzaldehyde, methyl 2-aminobenzoate, ethyl methylphenylglycidate, octalactone gamma, ethyl 3-phenyl-2-propenoate, (−)-(2E)-2-ethyl-4-[(1R)-2,2,3-trimethyl-3-cyclopenten-1-yl]-2-buten-1-ol, paracresyl acetate, dodecalactone, tricyclone, (+)-(3R,5Z)-3-methyl-5-cyclopentadecen-1-one, undecalactone, (1R,4R)-8-mercapto-3-p-menthanone, (3S,3AS,6R,7AR)-3,6-dimethylhexahydro-1-benzofuran-2 (3H)-one, beta ionone, (+−)-6-pentyltetrahydro-2H-pyran-2-one, (3E,5Z)-1,3,5-undecatriene, 10-undecenal, (9E)-9-undecenal (9Z)-9-undecenal, (Z)-4-decenal, (+−)-ethyl 2-methylpentanoate, 1,2-diallyldisulfane, 2-tridecenenitrile, 3-tridecenenitrile, (+−)-2-ethyl-4,4-dimethyl-1,3-oxathiane, (+)-(3R,5Z)-3-methyl-5-cyclopentadecen-1-one, 3-(4-tert-butylphenyl) propanal, allyl (cyclohexyloxy)acetate, methylnaphthylketone, (+−)-(4E)-3-methyl-4-cyclopentadecen-1-one, (+−)-5E3-methyl-5-cyclopentadecen-1-one, cyclopropylmethyl 3-hexenoate, (4E)-4-methyl-5-(4-methylphenyl)-4-pentenal, (+−)-1-(5-propyl-1,3-benzodioxol-2-yl) ethanone, 4-methyl-2-pentylpyridine, (+−)-(E)-3-methyl-4-(2,6,6-trimethyl-2-cyclohexen-1-yl)-3-buten-2-one, (3aRS,5aSR,9aSR,9bRS)-3a,6,6,9a-tetramethyldodecahydronaphtho [2,1-b]furan, (2S,5R)-5-methyl-2-(2-propanyl)cyclohexanone oxime, 6-hexyltetrahydro-2H-pyran-2-one, (+−)-3-(3-isopropyl-1-phenyl)butanal, methyl 2-(3-oxo-2-pentylcyclopentyl)acetate, 1-(2,6,6-trimethyl-1-cyclohex-2-enyl) pent-1-en-3-one, indol, 7-propyl-2H,4H-1,5-benzodioxepin-3-one, ethyl praline, (4-methylphenoxy)acetaldehyde, ethyl tricyclo[5.2.1.0.2.6]decane-2-carboxylate, (+)-(1'S,2S,E)-3,3-dimethyl-5-(2′,2′,3′-trimethyl-3′-cyclopenten-1′-yl)-4-penten-2-ol, (4E)-3,3-dimethyl-5-[(1R)-2,2,3-trimethyl-3-cyclopenten-1-yl]-4-penten-2-ol, 8-isopropyl-6-methyl-bicyclo[2.2.2]oct-5-ene-2-carbaldehyde, methylnonylacetaldehyde, 4-formyl-2-methoxyphenyl 2-methylpropanoate, (E)-4-decenal, (+−)-2-ethyl-4-(2,2,3-trimethyl-3-cyclopenten-1-yl)-2-buten-1-ol, (1R,5R)-4,7,7-trimethyl-6-thiabicyclo[3.2.1]oct-3-ene, (1R,4R,5R)-4,7,7-trimethyl-6-thiabicyclo[3.2.1]octane, (−)-(3R)-3,7-dimethyl-1,6-octadien-3-ol, (E)-3-phenyl-2-propenenitrile, 4-methoxybenzyl acetate, (E)-3-methyl-5-(2,2,3-trimethyl-3-cyclopenten-1-yl)-4-penten-2-ol, allyl (2/3-methylbutoxy)acetate, (+−)-(2E)-1-(2,6,6-trimethyl-2-cyclohexen-1-yl)-2-buten-1-one, (1E)-1-(2,6,6-trimethyl-1-cyclohexen-1-yl)-1-penten-3-one, and mixtures thereof.

According to an embodiment, perfume raw materials having a Log T<−4 are chosen in the group consisting of aldehydes, ketones, alcohols, phenols, esters lactones, ethers, epoxides, nitriles and mixtures thereof.

According to an embodiment, perfume raw materials having a Log T<−4 comprise at least one compound chosen in the group consisting of alcohols, phenols, esters lactones, ethers, epoxydes, nitriles and mixtures thereof, preferably in amount comprised between 20 and 70% by weight based on the total weight of the perfume raw materials having a Log T<−4. According to an embodiment, perfume raw materials having a Log T<−4 comprise between 20 and 70% by weight of aldehydes, ketones, and mixtures thereof based on the total weight of the perfume raw materials having a Log T<−4.

The remaining perfume raw materials contained in the oil-based core may have therefore a Log T>−4.

According to an embodiment, the perfume raw materials having a Log T>−4 are chosen in the group consisting of ethyl 2-methylbutyrate, (E)-3-phenyl-2-propenyl acetate, (+−)-6/8-sec-butylquinoline, (+−)-3-(1,3-benzodioxol-5-yl)-2-methylpropanal, verdyl propionate, 1-(octahydro-2,3,8,8-tetramethyl-2-naphtalenyl)-1-ethanone, methyl 2-((1RS,2RS)-3-oxo-2-pentylcyclopentyl)acetate, (+−)-(E)-4-methyl-3-decen-5-ol, 2,4-dimethyl-3-cyclohexene-1-carbaldehyde, 1,3,3-trimethyl-2-oxabicyclo[2.2.2]octane, tetrahydro-4-methyl-2-(2-methyl-1-propenyl)-2H-pyran, dodecanal, 1-oxa-12/13-cyclohexadecen-2-one, (+−)-3-(4-isopropylphenyl)-2-methylpropanal, aldehyde C11, (+−)-2,6-dimethyl-7-octen-2-ol, allyl 3-cyclohexylpropanoate, (Z)-3-hexenyl acetate, 5-methyl-2-(2-propanyl)cyclohexanone, allyl heptanoate, 2-(2-methyl-2-propanyl)cyclohexyl acetate, 1,1-dimethyl-2-phenylethyl butyrate, geranyl acetate, neryl acetate, (+−)-1-phenylethyl acetate, 1,1-dimethyl-2-phenylethyl acetate, 3-methyl-2-butenyl acetate, ethyl 3-oxobutanoate, (2Z)-ethyl 3-hydroxy-2-butenoate, 8-p-menthanol, 8-p-menthanyl acetate, 1-p-menthanyl acetate, (+−)-2-(4-methyl-3-cyclohexen-1-yl)-2-propanyl acetate, (+−)-2-methylbutyl butanoate, 2-{(1S)-1-[(1R)-3,3-dimethylcyclohexyl]ethoxy}-2-oxoethyl propionate, 3,5,6-trimethyl-3-cyclohexene-1-carbaldehyde, 2,4,6-trimethyl-3-cyclohexene-1-carbaldehyde, 2-cyclohexylethyl acetate, octanal, ethyl butanoate, (+−)-(3E)-4-(2,6,6-trimethyl-1/2-cyclohexen-1-yl)-3-buten-2-one, 1-[(1RS,6SR)-2,2,6-trimethylcyclohexyl]-3-hexanol, 1,3,3-trimethyl-2-oxabicyclo[2.2.2]octane, 1,3,3-trimethyl-2-oxabicyclo[2.2.2]octane, ethyl hexanoate, undecanal, decanal, 2-phenylethyl acetate, (1S,2S,4S)-1,7,7-trimethylbicyclo[2.2.1]heptan-2-ol, (1S,2R,4S)-1,7,7-trimethylbicyclo[2.2.1]heptan-2-ol), (+−)-3,7-dimethyl-3-octanol, 1-methyl-4-(2-propanylidene)cyclohexene, (+)-(R)-4-(2-methoxypropan-2-yl)-1-methylcyclohex-1-ene, verdyl acetate, (3R)-1-[(1R,6S)-2,2,6-trimethylcyclohexyl]-3-hexanol, (3S)-1-[(1R,6S)-2,2,6-trimethylcyclohexyl]-3-hexanol, (3R)-1-[(1S,6S)-2,2,6-trimethylcyclohexyl]-3-hexanol, (+)-(1S,1′R)-2-[1-(3′,3′-dimethyl-1′-cyclohexyl)ethoxy]-2-methylpropyl propanoate, and mixtures thereof.

According to an embodiment, the perfume formulation comprises
0 to 60 wt. % of a hydrophobic solvent (based on the total weight of the perfume formulation),
40 to 100 wt. % of a perfume oil (based on the total weight of the perfume formulation), wherein the perfume oil has at least two, preferably all of the following characteristics:
   at least 35%, preferably at least 40%, preferably at least 50%, more preferably at least 60% of perfuming ingredients having a log P above 3, preferably above 3.5,
   at least 20%, preferably at least 25%, preferably at least 30%, more preferably at least 40% of bulky materials of groups 1 to 6, preferably 3 to 6 as previously defined and
   at least 15%, preferably at least 20%, more preferably at least 25%, even more preferably at least 30% of high impact perfume materials having a Log T<−4,
      optionally, further hydrophobic active ingredients.

According to a particular embodiment, the perfume comprises 0 to 60 wt. % of a hydrophobic solvent.

According to a particular embodiment, the hydrophobic solvent is a density balancing material preferably chosen in the group consisting of benzyl salicylate, benzyl benzoate, cyclohexyl salicylate, benzyl phenylacetate, phenylethyl phenylacetate, triacetin, ethyl citrate, methyl and ethyl salicylate, benzyl cinnamate, and mixtures thereof.

In a particular embodiment, the hydrophobic solvent has Hansen Solubility Parameters compatible with entrapped perfume oil.

The term “Hansen solubility parameter” is understood refers to a solubility parameter approach proposed by Charles Hansen used to predict polymer solubility and was developed around the basis that the total energy of vaporization of a liquid consists of several individual parts. To calculate the “weighted Hansen solubility parameter” one must combine the effects of (atomic) dispersion forces, (molecular) permanent dipole-permanent dipole forces, and (molecular) hydrogen bonding (electron exchange). The weighted Hansen solubility parameter” is calculated as (δD²+δP²+δH²)^{0.5}, wherein δD is the Hansen dispersion value (also referred to in the following as the atomic dispersion fore), δP is the Hansen polarizability value (also referred to in the following as the dipole moment), and δH is the Hansen Hydrogen-bonding (“h-bonding”) value (also referred to in the following as hydrogen bonding). For a more detailed description of the parameters and values, see Charles Hansen, The Three Dimensional Solubility Parameter and Solvent Diffusion Coefficient, Danish Technical Press (Copenhagen, 1967).

Euclidean difference in solubility parameter between a fragrance and a solvent is calculated as (4*(δDₛₒₗᵥₑₙₜ−δD_{fragrance})²+(δPₛₒₗᵥₑₙₜ−δP_{fragrance})²+(δHₛₒₗᵥₑₙₜ−δH_{fragrance})²)^{0.5}, in which δDₛₒₗᵥₑₙₜ, δPₛₒₗᵥₑₙₜ, and δHₛₒₗᵥₑₙₜ, are the Hansen dispersion value, Hansen polarizability value, and Hansen h-bonding values of the solvent, respectively; and δD_{fragrance}, δP_{fragrance}, and δH_{fragrance} are the Hansen dispersion value, Hansen polarizability value, and Hansen h-bonding values of the fragrance, respectively.

In a particular embodiment, the perfume oil and the hydrophobic solvent have at least two Hansen solubility parameters selected from a first group consisting of: an atomic dispersion force (δD) from 12 to 20, a dipole moment (δP) from 1 to 8, and a hydrogen bonding (δH) from 2.5 to 11.

In a particular embodiment, the perfume oil and the hydrophobic solvent have at least two Hansen solubility parameters selected from a second group consisting of: an atomic dispersion force (δD) from 12 to 20, preferably from 14 to 20, a dipole moment (δP) from 1 to 8, preferably from 1 to 7, and a hydrogen bonding (δH) from 2.5 to 11, preferably from 4 to 11.

In a particular embodiment, at least 90% of the perfume oil, preferably at least 95% of the perfume oil, most preferably at least of 98% of the perfume oil has at least two Hansen solubility parameters selected from a first group consisting of: an atomic dispersion force (δD) from 12 to 20, a dipole moment (δP) from 1 to 8, and a hydrogen bonding (δH) from 2.5 to 11.

In a particular embodiment, the perfume oil and the hydrophobic solvent have at least two Hansen solubility parameters selected from a second group consisting of: an atomic dispersion force (δD) from 12 to 20, preferably from 14 to 20, a dipole moment (δP) from 1 to 8, preferably from 1 to 7, and a hydrogen bonding (δH) from 2.5 to 11, preferably from 4 to 11.

According to an embodiment, the perfuming formulation comprises a fragrance modulator (that can be used in addition to the hydrophobic solvent when present or as substitution of the hydrophobic solvent when there is no hydrophobic solvent).

Preferably, the fragrance modulator is defined as a fragrance material with
i. a vapor pressure of less than 0.0008 Torr at 22° C.;
ii. a clogP of 3.5 and higher, preferably 4.0 and higher and more preferably 4.5
iii. at least two Hansen solubility parameters selected from a first group consisting of: an atomic dispersion force from 12 to 20, a dipole moment from 1 to 7, and a hydrogen bonding from 2.5 to 11,
iv. at least two Hansen solubility parameters selected from a second group consisting of: an atomic dispersion force from 14 to 20, a dipole moment from 1 to 8, and a hydrogen bonding from 4 to 11, when in solution with a compound having a vapor pressure range of 0.0008 to 0.08 Torr at 22° C.

Preferably, as examples the following ingredients can be listed as modulators but the list in not limited to the following materials: alcohol C12, oxacyclohexadec-12/13-en-2-one, 3-[(2′,2′,3′-trimethyl-3′-cyclopenten-1′-yl)methoxy]-2-butanol, cyclohexadecanone, (Z)-4-cyclopentadecen-1-one, cyclopentadecanone, (8Z)-oxacycloheptadec-8-en-2-one, 2-[5-(tetrahydro-5-methyl-5-vinyl-2-furyl)-tetrahydro-5-methyl-2-furyl]-2-propanol, muguet aldehyde, 1,5,8-trimethyl-13-oxabicyclo[10.1.0]trideca-4,8-diene, (+−)-4,6,6,7,8,8-hexamethyl-1,3,4,6,7,8-hexahydrocyclopenta[g]isochromene, (+)-(1S,2S,3S,5R)-2,6,6-trimethylspiro[bicyclo[3.1.1]heptane-3,1′-cyclohexane]-2′-en-4′-one, oxacyclohexadecan-2-one, 2-{(1S)-1-[(1R)-3,3-dimethylcyclohexyl]ethoxy}-2-oxoethyl propionate, (+)-(4R,4aS,6R)-4,4a-dimethyl-6-(1-propen-2-yl)-4,4a,5,6,7,8-hexahydro-2 (3H)-naphthalenone, amylcinnamic aldehyde, hexylcinnamic aldehyde, hexyl salicylate, (1E)-1-(2,6,6-trimethyl-1-cyclohexen-1-yl)-1,6-heptadien-3-one, (9Z)-9-cycloheptadecen-1-one.

According to a particular embodiment, the hydrophobic material is free of any active ingredient (such as perfume). According to this particular embodiment, it comprises, preferably consists of hydrophobic solvents, preferably chosen in the group consisting of isopropyl myristate, tryglycerides (e.g. Neobee® MCT oil, vegetable oils), D-limonene, silicone oil, mineral oil, and mixtures thereof with optionally hydrophilic solvents preferably chosen in the group consisting of 1,4-butanediol, benzyl alcohol, triethyl citrate, triacetin, benzyl acetate, ethyl acetate, propylene glycol (1,2-propanediol), 1,3-propanediol, dipropylene glycol, glycerol, glycol ethers and mixtures thereof.

The term “biocide” refers to a chemical substance capable of killing living organisms (e.g. microorganisms) or reducing or preventing their growth and/or accumulation. Biocides are commonly used in medicine, agriculture, forestry, and in industry where they prevent the fouling of, for example, water, agricultural products including seed, and oil pipelines. A biocide can be a pesticide, including a fungicide, herbicide, insecticide, algicide, molluscicide, miticide and rodenticide; and/or an antimicrobial such as a germicide, antibiotic, antibacterial, antiviral, antifungal, antiprotozoal and/or antiparasite.

As used herein, a “pest control agent” indicates a substance that serves to repel or attract pests, to decrease, inhibit or promote their growth, development or their activity. Pests refer to any living organism, whether animal, plant or fungus, which is invasive or troublesome to plants or animals, pests include insects notably arthropods, mites, spiders, fungi, weeds, bacteria and other microorganisms.

According to any one of the invention's embodiments, the hydrophobic material represents between about 10 wt. % and 90 wt. %, preferably between 12 wt. % and 60 wt. %, or even more preferably between 15 wt. % and 45 wt. % relative to the total weight of the oil phase.

### Stabilizer

According to the present invention, a stabilizer is added in the oil phase in step a) and/or in the aqueous phase in step b).

A stabilizer is herein understood to be a compound suitable to stabilize oil-in-water emulsions. Stabilizers are herein also understood to be emulsifiers or dispersants.

According to one embodiment, the stabilizer is a colloidal stabilizer.

According to a preferred embodiment, the colloidal stabilizer is a polyvinyl alcohol or polyvinylpyrrolidone, or a mixture thereof.

According to an embodiment, the stabilizer is an anionic or amphiphilic biopolymer, preferably chosen from the group consisting of whey protein isolate, sodium caseinate, gum arabic, sugar beet pectin, gelatin, a protein and/or, and combinations thereof.

According to an embodiment, the stabilizer is a protein chosen in the group consisting of soy protein, hydrolyzed silk protein, potato protein, chickpea protein, pea protein, algae protein, faba bean protein, barley protein, oat protein, wheat gluten protein, lupin protein, and mixtures thereof.

According to a preferred embodiment, the stabilizer is a combination of sodium caseinate and whey protein isolate.

According to an embodiment, the stabilizer is a cationic stabilizer.

According to one embodiment, the stabilizer is combined with a salt. According to a preferred embodiment, the anionic or amphiphilic biopolymer is combined with a salt. According to an embodiment, the salt is chosen in the group consisting of calcium, sodium, potassium, lithium, magnesium, sulphates, phosphates, nitrates, bromides, chlorides, iodides, and ammonium salts. According to a preferred embodiment, the salt is chosen in the group consisting of CaCl₂), NaCl, KCl, LiCl, Ca(NO₃)₂, MgCl₂, CaBr₂, CaI₂, NaBr, NaI, NaNO₃, KBr, KI, KNO₃, LiBr, LiI, MgBr₂ and mixtures thereof. According to a more preferred embodiment, the salt is chosen in the group consisting of CaCl₂), NaCl, KCl, LiCl, Ca (NO₃)₂, MgCl₂, and mixtures thereof.

Even more preferred, the stabilizer is selected from the group of polyvinyl alcohol, whey protein isolate, sodium caseinate, cellulose polymers, polyvinyl pyrrolidone, anionic polyelectrolytes and/or bovine serum albumin.

According to a particular embodiment, the stabilizer is an aqueous phase of 0.1 wt. % to 8.0 wt. %, preferably of 0.5 wt. % to 4.0 wt. %, more preferably of 1.0 wt. % to 2.0 wt. % of polyvinyl alcohol.

According to a particular embodiment, the stabilizer is dispersed directly in the oil phase.

According to a particular embodiment, the stabilizer is an aqueous phase of 0.01 wt. % to 2.0 wt. % calcium chloride dihydrate, 0.1 wt. % to 5.0 wt. % sodium caseinate and 0.1 wt. % to 5.0 wt. % of whey protein isolate.

According to a preferred embodiment, the stabilizer is an aqueous phase of 0.1 wt. % to 1.5 wt. % calcium chloride dihydrate, 0.2 wt. % to 3.0 wt. % sodium caseinate and 0.2 wt. % to 3.0 wt. % of whey protein isolate.

According to a more preferred embodiment, the stabilizer is an aqueous phase of 0.25 wt. % to 0.75 wt. % calcium chloride dihydrate, 0.43 wt. % to 2.0 wt. % sodium caseinate and 0.3 wt. % to 2.0 wt. % of whey protein isolate.

According to another embodiment, the stabilizer is a surfactant. A surfactant is herein understood to be a compound suitable to lower the surface tension or interfacial tension between the oil phase and the aqueous phase. A surfactant is herein understood to be an amphiphilic organic compound comprising a hydrophilic, or water-soluble, part and a hydrophobic, or oil-soluble, part.

According to one embodiment, the stabilizer is present in an amount of 0.1 wt. % to 20 wt. %, preferably 0.3 wt. % to 7 wt. %, more preferably 0.5 wt. % to 5 wt. %, based on the total weight of the emulsion obtained in step b) of the process of the invention.

According to an embodiment, the dispersion of the oil phase in the aqueous phase is homogenized at a speed of 2000 rpm to 20,000 rpm, preferably 6000 rpm to 12000 rpm for a duration of 30 seconds to 10 minutes, preferably 1 minute to 5 minutes. A person skilled in the art is aware of suitable ways for homogenizing the oil-in-water dispersion, e.g. by using a T25 Ultra Turrax homogenizer.

According to an embodiment, the oil phase is a water-in-oil emulsion comprising dispersed droplets of a second aqueous phase. According to a particular embodiment, the second aqueous phase contains water and at least one water-soluble oxidant and/or oxidation catalyst as defined herein.

### Oxidant

According to the invention, an oxidant is added in the aqueous phase in step b) and/or in the oil-in-water emulsion obtained after step b).

According to an embodiment, an oxidant is added in the oil-in-water emulsion under stirring to induce thiol oxidation at the oil-water interface.

An oxidant is herein understood to be a redox active compound. An oxidant is herein understood to be a redox active compound suitable to oxidize thiol groups to disulfide groups. A person skilled in the art is aware of the fact that a disulfide group is formed by oxidation of two thiol groups by reaction with an oxidant.

According to an embodiment, the oxidant is an inorganic redox active compound. According to another embodiment, the oxidant is an organic redox active compound.

According to an embodiment, the oxidant is iodine, iodide salt, iron (II) sulfate, hydrogen peroxide, iodobenzene diacetate, ammonium persulfate, potassium ferricyanide, ammonium perborate, iron (III) chloride, potassium dichromate, sodium bromide, potassium bromide, bromine, triiodide salt, copper (II) sulfate, N-bromosuccinimide, or a mixture thereof. According to a preferred embodiment, the oxidant is hydrogen peroxide.

According to an embodiment, the oxidant is iodine, iodide salt, iron (II) sulfate, hydrogen peroxide, iodobenzene diacetate, ammonium persulfate, potassium ferricyanide, ammonium perborate, ferric chloride, potassium dichromate, potassium bromide, bromine, triiodide salt, copper (II) sulfate, N-bromosuccinimide, or mixtures thereof, preferably hydrogen peroxide.

According to an embodiment, the oxidant is used in a molar ratio of thiol functional groups to oxidant of 1:10 to 1:0.01, preferably of 1:6 to 1:0.5, more preferably of 1:2 to 1:1.

### Oxidation Catalyst

According to the invention, an oxidation catalyst is used in combination with the oxidant to oxidize thiol groups to disulfide groups. According to the invention, the oxidation catalyst is suitable to catalyze the oxidation reaction of thiol groups to disulfide groups, preferably at the oil-water interface.

According to the invention, the oxidation catalyst is added in the oil phase in step a) and/or in the oil-in-water emulsion obtained after step b).

According to an embodiment, the oxidation catalyst is added in the oil phase in step a). The oxidation catalyst added in the oil phase in step a) can optionally be dissolved in a co-solvent.

According to an embodiment, the oxidation catalyst is dissolved in a co-solvent and is added in the oil phase in step a).

According to an embodiment, the oxidation catalyst is added in the oil-in-water emulsion obtained after step b). The oxidation catalyst added in the oil-in-water emulsion obtained after step b) can optionally be dissolved in water.

According to another embodiment, the oxidation catalyst is dissolved in water and added in the oil-in-water emulsion obtained after step b).

According to an embodiment, a first oxidation catalyst, optionally dissolved in a co-solvent, is added in the oil phase in step a) and a second oxidation catalyst, optionally dissolved in water, is added in the oil-in-water emulsion obtained after step b).

According to a preferred embodiment, the first oxidation catalyst and the second oxidation catalyst are different oxidation catalysts.

According to an embodiment, the first oxidation catalyst and the second oxidation catalyst are the same oxidation catalyst and added in the oil phase in step a) and in the oil-in-water emulsion obtained after step b).

According to an embodiment, the oxidation catalyst is iodine, iodobenzene diacetate, a bromide salt, or an iodide salt. The iodide salt can be in the form of an n-hydrate, wherein n ranges from 1 to 10 (for example for n=2, it is a dihydrate iodide salt).

According to a preferred embodiment, the oxidation catalyst is an iodide salt selected from the group of sodium iodide, potassium iodide, tridodecylmethylammonium iodide, ethyltriphenylphosphonium iodide, propionylthiocholine iodide, isopropyltriphenylphosphonium iodide, tin(IV) iodide, aluminum iodide, acetylthiocholine iodide, butyrylcholine iodide, butyrylthiocholine iodide, tetraethylammonium iodide, phenyltriethylammonium iodide, methyltriphenoxyphosphonium iodide, manganese(II) iodide, iron(II) iodide, zinc iodide, perfluorohexyl iodide, tetramethylammonium iodide, nickel(II) iodide, magnesium iodide, ruthenium iodide, ammonium iodide, tributyltin iodide, lithium iodide, methyltriphenylphosphonium iodide, trimethylsulfonium iodide, tetrapentylammonium iodide, tetraphenylphosphonium iodide, acetylcholine iodide, copper(I) iodide, lead(II) iodide, strontium iodide, cadmium iodide, tetrabutylammonium iodide, calcium iodide, rubidium iodide, cesium iodide, palladium(II) iodide, (iodomethyl)triphenylphosphonium iodide, arsenic(III) iodide, tetrahexylammonium iodide, benzyltributylammonium iodide, bis[(tetrabutylammonium iodide)copper(I) iodide], 3,3′-diethylthiatricarbocyanine iodide, methylammonium iodide, formamidinium iodide, 1-dodecyl-3-methylimidazolium iodide, 1-aminopyridinium iodide, tetraheptylammonium iodide, guanidinium iodide, n-octylammonium iodide, tetrahexylammonium iodide, 1,4-dimethylpyridinium iodide, 3,3′-diethylthiacarbocyanine iodide, 1-butyl-3-methylimidazolium iodide, 1-methyl-3-propylimidazolium iodide, N,N-dimethylmethyleneiminium iodide, 2-chloro-1-methylpyridinium iodide, (N,N-dimethyl)methyleneammonium iodide, 1-ethylquinolinium iodide, benzoylcholine lodide, trimethylphenylammonium iodide, triethylphenylammonium lodide, 2-phenylethylamine hydroiodide, N,N-dimethyl-N-(methylsulfanylmethylene) ammonium iodide, dimethylamine hydroiodide, isobutylamine hydroiodide, tributylsulfonium iodide, butylamine hydroiodide, tert-butylamine hydroiodide, 1-ethyl-2-methylquinolinium iodide, aniline hydroiodide, pyrrolidine hydriodide, propylamine hydroiodide, phenyltrimethylammonium iodide, 1,1-dimethyl-4-phenylpiperazinium iodide, 3-methylbenzothiazolium iodide, chlorisondamine diiodide, 3,3′-diethyloxacarbocyanine iodide, 3,3′-dihexyloxacarbocyanine iodide, 3,3′-diethylthiatricarbocyanine iodide, 1,1′-diethyl-2,2′-carbocyanine iodide, 3,3′-diethylthiadicarbocyanine iodide or mixtures thereof. According to a preferred embodiment, the oxidation catalyst is sodium iodide.

According to an embodiment, the oxidation catalyst and the oxidant are the same compound. In said embodiment, the same compound thus acts as both oxidation catalyst and oxidant at the same time.

According to an embodiment, the oxidation catalyst and the oxidant are different compounds.

According to an embodiment, the oxidation catalyst is used in a molar ratio of thiol functional groups to oxidation catalyst of 1:1 to 1:0.0001, preferably of 1:0.5 to 1:0.005, more preferably of 1:0.1 to 1:0.01.

### Co-Solvent

According to an embodiment, a co-solvent is used to dissolve an oxidation catalyst that is added in the oil phase in step a).

Without wishing to be bound to any theory, the co-solvent increases the solubility of the oxidation catalyst in the oil phase.

According to an embodiment, the co-solvent is an organic solvent. An organic solvent is herein be understood to be a solvent soluble in the oil phase of the step a) of the process of the invention. According to an embodiment, the organic solvent is polar and hydrophilic. According to another embodiment, the organic solvent is nonpolar and hydrophobic.

According to a preferred embodiment, the co-solvent is ethyl acetate, ethanol, isopropanol alcohol, dimethyl sulfoxide, acetone, glycerol or a perfumery ingredient, preferably benzylbezoate or isopropylmyristate, or mixtures thereof.

According to an embodiment, the co-solvent is used in an amount of 0.01 wt. % to 30 wt. %, preferably 0.5 wt. % to 15 wt. %, more preferably 1.0 wt. % to 5 wt. %, based on the total weight of the emulsion obtained in step b).

In a particular embodiment, the shell material comprises a biodegradable material.

A typical method for extracting the shell for measuring the biodegradability is disclosed in Gasparini et al., Molecules 2020, 25 (3), 718.

In a particular embodiment, the shell has a biodegradability of at least 40%, preferably at least 45%, 50%, 60%, 65%, 70%, 75%, 80%, 85%, 90%, 95% or 98%, within 60 days according to OECD301F.

In a particular embodiment, the core-shell microcapsule has a biodegradability of at least 40%, preferably at least 60%, preferably at least 65%, 70%, 75%, 80%, 85%, 90%, 95% or 98% within 60 days according to OECD301F.

Thereby it is understood that, according to an embodiment, the core-shell microcapsule including all components, such as the core, shell and optionally coating have a biodegradability of at least 40%, preferably at least 60%, preferably at least 65%, 70%, 75%, 80%, 85%, 90%, 95% or 98% within 60 days according to OECD301F.

In a particular embodiment, the oil core, preferably perfume oil, has a biodegradability of at least 40%, preferably at least 60%, preferably at least 65%, 70%, 75%, 80%, 85%, 90%, 95% or 98% within 60 days according to OECD301F.

OECD301F is a standard test method on the biodegradability from the Organization of Economic Co-operation and Development.

### Drying Step

According to an embodiment, the process of the invention comprises an additional drying step d) wherein the polydisulfide core-shell microcapsule slurry is dried to form a polydisulfide core-shell microcapsule powder.

A person skilled in the art is aware of suitable methods for producing a polydisulfide core-shell microcapsule powder by drying a polydisulfide core-shell microcapsule slurry. Non-limiting examples for drying are spray drying or freeze drying.

According to a preferred embodiment, the polydisulfide core-shell microcapsule powder is produced by spray drying a polydisulfide core-shell microcapsule slurry.

Another object of the invention is a solid particle comprising:
a carrier material, preferably a polymeric carrier material chosen in the group consisting of polyvinyl acetate, polyvinyl alcohol, dextrins, natural or modified starch, vegetable gums such as gum arabic, pectins, xanthans, alginates, carragenans, cellulose derivatives and mixtures thereof, and
microcapsules as defined above entrapped in said carrier material, and
optionally free perfume entrapped in said carrier material.

Solid particle as defined above and microcapsule powder can be used indifferently in the present invention.

### Process Parameters

According to an embodiment, the reaction temperature in step c) is kept between 0° C. and 70° C., preferably between 10° C. and 65° C., more preferably between 15° C. and 60° C.

According to an embodiment, the pH is adjusted to 1 to 12, preferably to 3 to 8, in step b) and/or step c).

According to an embodiment, the mixture of step c) is stirred for a reaction time of 1 minute to 48 hours, preferably of 10 minutes to 24 hours.

### Optional Components

According to one embodiment, the microcapsule slurry can comprise auxiliary ingredients selected from the group of thickening agents/rheology modifiers, antimicrobial agents, opacity-building agents, mica particles, salt, pH stabilizers/buffering ingredients, preferably in an amount comprised between 0.1 wt. % and 15 wt. %, preferably between 5 wt. % and 10 wt. %, based on the total weight of the slurry.

According to another embodiment, the microcapsule slurry of the invention comprises additional free (i.e non-encapsulated) perfume, preferably in an amount comprised between 5 wt. % and 50 wt. %, preferably between 15 wt. % and 35 wt. %, based on the total weight of the slurry.

### Polydisulfide Core-Shell Microcapsules

The invention is also directed to polydisulfide core-shell microcapsules as prepared by the process of the invention, comprising
an oil-based core comprising a hydrophobic material, preferably a perfume oil; and
a polymeric shell comprising disulfide bonds formed from the oxidation from at least one polythiol monomer with an oxidant in the presence of an oxidation catalyst.

The embodiments and definitions for the oil-based core comprising a hydrophobic material, disulfide bonds, polythiol monomers, oxidant and oxidation catalyst as described herein-above applies mutatis mutandis.

According to one embodiment, the polydisulfide core-shell microcapsules are in liquid form of a polydisulfide core-shell microcapsule slurry.

According to one embodiment, the polydisulfide core-shell microcapsules are in powdery form of a polydisulfide core-shell microcapsule powder.

According to the invention, the polymeric shell is a shell comprising at least one polymer forming a surrounding structure around the oil-based core.

According to the invention, the polymeric shell of the polydisulfide core-shell microcapsules comprises at least one polymer comprising disulfide bonds. Disulfide bonds are herein understood to be sulfur-sulfur single bonds. According to the invention, the polymeric shell of the polydisulfide core-shell microcapsules is formed by formation of disulfide linkages upon oxidation of at least one polythiol monomer.

According to one embodiment, the disulfide bonds are crosslinking the polymeric shell of the polydisulfide core-shell microcapsules.

### Biomineralization Step

The microcapsules of the present invention can be coated with a mineral layer. Typically, the mineral layer comprises a material chosen in the group consisting of iron oxides, iron oxyhydroxide, titanium oxides, zinc oxides, calcium carbonates, calcium phosphates and mixtures thereof.

### Multiple Microcapsule System

According to an embodiment, the microcapsules of the invention (first type of microcapsule) can be used in combination with a second type of microcapsules.

Another object of the invention is a microcapsule delivery system comprising:
the microcapsules of the present invention as a first type of microcapsule, and
a second type of microcapsules, wherein the first type of microcapsule and the second type of microcapsules differ in their hydrophobic material and/or their wall material and/or in their coating material.

### Composition

The microcapsules of the invention can be used in combination with active ingredients. An object of the invention is therefore a composition comprising:
(i) microcapsules of the present invention;
(ii) an active ingredient, preferably chosen in the group consisting of a cosmetic ingredient, skin caring ingredient, perfume ingredient, flavor ingredient, malodour counteracting ingredient, bactericide ingredient, fungicide ingredient, pharmaceutical or agrochemical ingredient, a sanitizing ingredient, an insect repellent or attractant, and mixtures thereof.

### Perfuming Composition

The present invention also relates to a perfuming composition comprising polydisulfide core-shell microcapsules as defined herein, at least one ingredient selected from the group consisting of a perfumery carrier and a perfumery base and optionally at least one active ingredient and optionally at least one perfumery adjuvant.

The embodiments and definitions for the polydisulfide core-shell microcapsules, polymeric shell, oil-based core comprising a hydrophobic material, disulfide bonds, polythiol monomers, oxidant and oxidation catalyst as described herein-above applies mutatis mutandis.

The perfuming composition may comprise the core-shell microcapsule slurry or core-shell microcapsules between 0.1 wt. % and 30 wt. %, preferably between 0.3 wt. % to 15 wt. %, more preferably between 0.5 wt. % to 5 wt. %, based on the total weight of the perfuming composition.

The perfumery carrier may be a liquid perfumery carrier. As liquid perfumery carrier, one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples solvents such as dipropyleneglycol, diethyl phthalate, isopropyl myristate, benzyl benzoate, 2-(2-ethoxyethoxy)-1-ethanol or ethyl citrate, which are the most commonly used.

For the compositions which comprise both a perfumery carrier and a perfumery co-ingredient, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company).

The perfuming composition may further comprise an active ingredient. The active ingredient may preferably be chosen in the group consisting of a cosmetic ingredient, skin caring ingredient, perfume ingredient, flavor ingredient, malodor counteracting ingredient, bactericide ingredient, fungicide ingredient, pharmaceutical or agrochemical ingredient, a sanitizing ingredient, an insect repellent or attractant, and mixtures thereof.

In a particular embodiment, the active ingredient is a free perfume. The free perfume may be a free perfume oil.

By “free perfume” it is herein understood a perfume or perfume oil which is comprised in the perfuming composition and not entrapped in the polydisulfide core-shell microcapsule.

The perfuming composition may comprise the active ingredient, preferably the free perfume, between 0.1 wt. % and 30 wt. %, preferably between 0.3 wt. % to 15 wt. %, more preferably between 0.5 wt. % to 5 wt. %, based on the total weight of the perfuming composition.

In a particular embodiment, the total amount of the polydisulfide core-shell microcapsule slurry or polydisulfide core-shell microcapsule is 0.5 wt. % to 5 wt. %, based on the total weight of the perfuming composition, and the total amount of the free perfume is 0.5 wt. % to 5 wt. %, based on the total weight of the perfuming composition.

In a particular embodiment, the total amount of perfume of the perfume formulation entrapped in the polydisulfide core-shell microcapsule and total free perfume are present in a weight ratio of 1:20 to 20:1, preferably 10:1 to 1:10 in the perfuming composition.

According to an embodiment, the perfuming composition further comprises at least one perfuming co-ingredient.

By “perfuming co-ingredient” it is herein understood a compound, which is used in a perfuming preparation or a composition to impart a hedonic effect and which is not a microcapsule as defined above. In other words, such a co-ingredient, to be considered as being a perfuming one, must be recognized by a person skilled in the art as being able to impart or modify in a positive or pleasant way the odor of a composition, and not just as having an odor. The nature and type of the perfuming co-ingredients present in the perfuming composition do not warrant a more detailed description here, which in any case would not be exhaustive, the skilled person being able to select them based on his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said co-ingredients may also be compounds known to release in a controlled manner various types of perfuming compounds (such as pro-perfumes). Non-limiting examples of suitable properfumes may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, trans-3-(dodecylthio)-1-(2,6,6-trimethyl-3-cyclohexen-1-yl)-1-butanone, 2-(dodecylthio) octan-4-one, 2-phenylethyl oxo (phenyl)acetate, 3,7-dimethylocta-2,6-dien-1-yl oxo (phenyl)acetate, (Z)-hex-3-en-1-yl oxo (phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy) undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-4-((2-methoxy-2-phenylvinyl)oxy)benzaldehyde, 4-((2-(hexyloxy)-2-phenylvinyl)oxy)-3-methoxybenzaldehyde or a mixture thereof.

According to an embodiment, the perfuming composition further comprises at least one perfumery adjuvant.

By “perfumery adjuvant” it is herein understood an ingredient capable of imparting additional added benefit such as a color, a particular light resistance, chemical stability, etc. A detailed description of the nature and type of adjuvant commonly used in perfuming bases cannot be exhaustive, but it has to be mentioned that said ingredients are well known to a person skilled in the art.

### Consumer Product

The polydisulfide core-shell microcapsules according to the invention can advantageously be used in many application fields and used in consumer products.

The polydisulfide core-shell microcapsules or the perfuming composition can be used in liquid form applicable to liquid consumer products as well as in powder form, applicable to powder consumer products.

The present invention also relates to a consumer product comprising a personal care active base or a fabric care active base and polydisulfide core-shell microcapsules or a perfuming composition.

The embodiments and definitions for the polydisulfide core-shell microcapsules, polymeric shell, oil-based core comprising a hydrophobic material, disulfide bonds, polythiol monomers, oxidant, oxidation catalyst, perfumery carrier, perfuming composition, perfumery base, perfuming co-ingredient and perfumery adjuvant as described herein-above applies mutatis mutandis.

According to an embodiment, the consumer product is in the form of a personal care composition, home care composition or fabric care composition, preferably in form of a hair care product, such as a shampoo or hair-conditioner, a shower gel, a soap bar, a lotion, an antiperspirant, such as an antiperspirant roll on stick or antiperspirant deodorant, an oral care product, a laundry care product, such as a rinse-off conditioner, leave-on conditioner, fabric softener, a liquid or solid detergent, preferably a liquid fabric detergent or powdered fabric detergent, most preferably in the form of a shower gel or a fabric softener.

A personal care active base or a fabric care active base in which the polydisulfide core-shell microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

According to an embodiment, the polydisulfide core-shell microcapsules or the perfuming composition can be dispersed in the personal care active base or fabric care active base without visibly affecting the color of the personal care active base or fabric care active base.

According to an embodiment, the consumer product comprises:
a) from 2 to 65% by weight, relative to the total weight of the consumer product, of at least one surfactant;
b) water or a water-miscible hydrophilic organic solvent;
c) microcapsules or a microcapsule slurry as defined above,
d) optionally non-encapsulated perfume.

According to an embodiment, the consumer product comprises from 0.1 to 15 wt %, more preferably between 0.2 and 5 wt % of the microcapsules of the present invention, these percentages being defined by weight relative to the total weight of the consumer product. Of course, the above concentrations may be adapted according to the benefit effect desired in each product.

For liquid consumer product mentioned below, by “active base”, it should be understood that the active base includes active materials (typically including surfactants) and water.

For solid consumer product mention below, by “active base”, it should be understood that the active base includes active materials (typically including surfactants) and auxiliary agents (such as bleaching agents, buffering agent; builders; soil release or soil suspension polymers; granulated enzyme particles, corrosion inhibitors, antifoaming, sud suppressing agents; dyes, fillers, and mixtures thereof).

### Fabric Softener

An object of the invention is a consumer product in the form of a fabric softener composition comprising:
a fabric softener active base; preferably comprising at least one active material chosen in the group consisting of dialkyl quaternary ammonium salts, dialkyl ester quaternary ammonium salts (esterquats), Hamburg esterquat (HEQ), TEAQ (triethanolamine quat), silicones and mixtures thereof, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Liquid Detergent

An object of the invention is a consumer product in the form of a liquid detergent composition comprising:
a liquid detergent active base; preferably comprising at least one active material chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly (ethylene glycol) ether, fatty alcohol ethoxylate (FAE), ethylene oxide (EO) and propylene oxide (PO) copolymers, amine oxydes, alkyl polyglucosides, alkyl polyglucosamides, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition, optionally free perfume oil.

### Solid Detergent

An object of the invention is a consumer product in the form of a solid detergent composition comprising:
a solid detergent active base; preferably comprising at least one active material chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, the active base being used preferably in an amount comprised between 85 and 99.95% by weight based on the total weight of the composition,
a microcapsule powder or microcapsule slurry or microcapsules as defined above, preferably in an amount comprised between 0.05 to 15 wt %, more preferably between 0.1 and 5 wt % by weight based on the total weight of the composition,
optionally free perfume oil.

### Shampoo/Shower Gel

An object of the invention is a consumer product in the form of a shampoo or a shower gel composition comprising:
a shampoo or a shower gel active base; preferably comprising at least one active material chosen in the group consisting of sodium alkylether sulfate, ammonium alkylether sulfates, alkylamphoacetate, cocamidopropyl betaine, cocamide MEA, alkylglucosides and aminoacid based surfactants and mixtures thereof, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Rinse-Off Conditioner

An object of the invention is a consumer product in the form of a rinse-off conditioner composition comprising:
a rinse-off conditioner active base; preferably comprising at least one active material chosen in the group consisting of cetyltrimonium chloride, stearyl trimonium chloride, benzalkonium chloride, behentrimonium chloride and mixture thereof, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Solid Scent Booster

An object of the invention is a consumer product in the form of a solid scent booster composition comprising:
a solid carrier, preferably chosen in the group consisting of urea, sodium chloride, sodium sulphate, sodium acetate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, saccharides such as sucrose, mono-, di-, and polysaccharides and derivatives such as starch, cellulose, methyl cellulose, ethyl cellulose, propyl cellulose, polyols/sugar alcohols such as sorbitol, maltitol, xylitol, erythritol, and isomalt, PEG, PVP, citric acid or any water soluble solid acid, fatty alcohols or fatty acids and mixtures thereof,
microcapsules or a microcapsule slurry as defined above, preferably in a powdered form, preferably in an amount comprised between 0.05 to 15 wt %, more preferably between 0.1 and 5 wt % by weight based on the total weight of the composition,
optionally free perfume oil.

### Liquid Scent Booster

An object of the invention is a consumer product in the form of a liquid scent booster composition comprising:
an aqueous phase,
a surfactant system essentially consisting of one or more than one non-ionic surfactant, wherein the surfactant system has a mean HLB between 10 and 14, preferably chosen in the group consisting of ethoxylated aliphatic alcohols, POE/PPG (polyoxyethylene and polyoxypropylene) ethers, mono and polyglyceryl esters, sucrose ester compounds, polyoxyethylene hydroxylesters, alkyl polyglucosides, amine oxides and combinations thereof;
a linker chosen in the group consisting of alcohols, salts and esters of carboxylic acids, salts and esters of hydroxyl carboxylic acids, fatty acids, fatty acid salts, glycerol fatty acids, surfactant having an HLB less than 10 and mixtures thereof, and
a microcapsule slurry or microcapsules as defined above, in the form of a slurry, preferably in an amount comprised between 0.05 to 15 wt %, more preferably between 0.1 and 5 wt % by weight based on the total weight of the composition,
optionally free perfume oil.

### Hair Coloration

An object of the invention is a consumer product in the form of an oxidative hair coloring composition comprising:
an oxidizing phase comprising an oxidizing agent and an alkaline phase comprising an alkakine agent, a dye precursor and a coupling compound; wherein said dye precursor and said coupling compound form an oxidative hair dye in the presence of the oxidizing agent, preferably in an amount comprised between 85 and 99.95% by weight based on the total weight of the composition,
microcapsules or a microcapsule slurry as defined above, preferably in an amount comprised between 0.05 to 15 wt %, more preferably between 0.1 and 5 wt % by weight based on the total weight of the composition,
optionally free perfume oil

### Perfuming Composition

According to a particular embodiment, the consumer product is in the form of a perfuming composition comprising:
0.1 to 30%, preferably 0.1 to 20% of microcapsules or a microcapsule slurry, preferably in the form of a slurry as defined previously,
0 to 40%, preferably 3-40% of perfume, and
20-90%, preferably 40-90% of ethanol, by weight based on the total weight of the perfuming composition.

The invention will now be further described by way of examples. It will be appreciated that the invention as claimed is not intended to be limited in any way by these examples.

### EXAMPLES

### Example 1: Process for Preparing Polydisulfide Core-Shell Microcapsules

### Perfume Compositions Used:

TABLE 1
Perfume oil 1 composition

| Ingredients | % in oil |
|---|---|
| Cyclosal¹⁾ | 25% |
| Tert-butyl-1-cyclohexyl acetate²⁾ | 25% |
| Romascone³⁾ | 25% |
| Salicynile⁴⁾ | 25% |
| Dorisyl⁵⁾ | 25% |

¹⁾3-(4-Isopropylphenyl)-2-methylpropanal
²⁾Tert-butyl-1-cyclohexyl acetate
³⁾Methyl 2,2-dimethyl-6-methylene-1-cyclohexanecarboxylate
⁴⁾(2Z)-2-Phenyl-2-hexenenitrile
⁵⁾4-(Tert-Butyl) cyclohexyl acetate

TABLE 2
Perfume oil 2 composition

| Ingredients | % in oil |
|---|---|
| Ethyl 2-methyl-pentanoate | 3.2% |
| Eucalyptol | 7.8% |
| 2,4-Dimethyl-3-cyclohexene-1-carbaldehyde | 0.75% |
| Aldehyde C10 | 0.75% |
| Citronellyl Nitrile | 4.3% |
| Isobornyl acetate | 3% |
| 2-tert-butyl-1-cyclohexyl acetate | 9.8% |
| Citronellyl Acetate | 1.3% |
| 2-Methylundecanal | 3% |
| Diphenyloxide | 0.8% |
| Aldehyde C12 | 1.3% |
| Dicyclopentadiene acetate | 9.85% |
| Ionone beta | 3.3% |
| Undecalactone gamma | 18.75% |
| Hexyl Salicylate | 15.9% |
| Benzyl Salicylate | 16.2% |

The following reactants are used for the preparation of disulfide microcapsules:
Ethylene glycol bis(3-mercaptopropionate) is used as dithiol monomer.
Tris[2-(3-mercaptopropionyloxy)ethyl] isocyanurate is used as trithiol monomer.
Pentaerythritol hexakis (3-mercaptopropionate) is used as hexahiol monomer.

Room temperature is herein understood as a temperature between 20° C. and 25° C. or between 293 K and 298 K.

### Example 1B

The oil phase was obtained by dissolving 1.8 g of hexathiol monomer in 18 g Perfume oil composition 1 at room temperature upon vortex for 1 min. 0.85 g polyvinyl alcohol (PVA, Mowiol 18-88) was added to 60 g deionized (DI) water and dissolved after 1 h stirring at 70° C. The oil phase was then added to the PVA/DI water phase and mixed by homogenization at 8400 rpm for 2 min. Once the oil-in-water emulsion was obtained, 0.21 g NaI dissolved in minimal DI water was added to the oil-in-water emulsion stirring. 1.6 g hydrogen peroxide (H₂O₂ (30 wt. % aq.)) were then added to oil-in-water emulsion by two separate times (0.8 g each with a 30-min interval). The slurry was then stirred overnight at room temperature

### Example 1F

The oil phase was obtained by dissolving 1 g dithiol monomer and 1.08 g hexathiol monomer in 18 g Perfume oil composition 1 at room temperature upon vortex for 1 min. 0.85 g polyvinyl alcohol (PVA, Mowiol 18-88) was added to 60 g deionized (DI) water and dissolved after 1 h stirring at 70° C. The oil phase was then added to the PVA/DI water phase and mixed by homogenization at 8400 rpm for 2 min. Once the oil-in-water emulsion was obtained, 0.25 g NaI dissolved in minimal DI water was added to the oil-in-water emulsion and 1.9 g hydrogen peroxide (H₂O₂ (30 wt. % aq.) were added by two separate times with a 30-min interval. The slurry was heated at 40° C. for 3 h and then stirred overnight at room temperature.

### Example 1G

Oil phase I was prepared by dissolving 3.66 g trithiol monomer in 12 g Perfume oil composition 1 and 6 g Tert-butyl-1-cyclohexyl acetate, and oil phase II was prepared by adding 0.36 g NaI in 1.2 g IPOH. Oil phase II was then added into the oil phase I upon swirling. The water phase contains 0.9 g polyvinyl alcohol (PVA, Mowiol 18-88) in 60 g DI water. 1.35 g H₂O₂ (30 wt. % aq.) was pre-added to the water phase. The oil phase and water phase were then homogenized at 8400 rpm for 2 min to form oil-in-water emulsion. Another 1.35 g H₂O₂ (30 wt. % aq.) was then added to the oil-in-water emulsion. The microcapsules were obtained after overnight reaction at room temperature.

### Example 1H

The oil phase was obtained by dissolving 3.6 g trithiol monomer in 18 g Perfume oil composition 1 at room temperature upon vortex for 1 min. 1.7 g whey protein isolate (WPI) was added to 60 g deionized (DI) water and dissolved after 30 min stirring at 50° C. The oil phase was then added to the WPI/DI water phase and mixed by homogenization at 10000 rpm for 2 min. Once the oil-in-water emulsion was obtained, 0.3 g NaI dissolved in minimal DI water was added to the oil-in-water emulsion while stirring, and 2.3 g hydrogen peroxide (H₂O₂ (30 wt. % aq.)) was added to the slurry by two separate times with a 30 min interval. The microcapsules were obtained after overnight reaction at room temperature.

### Example 11

Oil phase I was prepared by dissolving 3.6 g trithiol monomer in 18 g Perfume oil composition 1, and oil phase II was prepared by adding 0.3 g NaI in 1.7 g ethyl acetate. Oil phase II was then added into the oil phase I upon swirling. The water phase contains 1.7 g whey protein isolate (WPI) in 60 g DI water. The oil phase and water phase were then mixed and homogenized at 10000 rpm for 2 min to form the oil-in-water emulsion. 2.3 g H₂O₂ (30 wt. % aq.) was then added to the oil-in-water emulsion separately twice with a 30-min time interval. The microcapsules were obtained after overnight reaction at room temperature.

### Example 1K

The oil I phase was obtained by dissolving 1.8 g of hexathiol monomer in 18 g Perfume oil composition 1 at room temperature upon vortex for 1 min. Oil Phase II comprises 0.1 g NaI in 0.4 g ethanol. The two oil phases were mixed with each other. 0.85 g PVA (Mowiol 18-88) was added to 60 g deionized (DI) water and dissolved after 1 h stirring at 70° C. The oil phase was then added to the PVA/DI water phase and mixed by homogenization at 8400 rpm for 2 min. 1.6 g hydrogen peroxide (H₂O₂ (30 wt. % aq.)) were added to the emulsion by two separate times (add half each time with a 30 min interval). The reaction was stopped after overnight stirring at room temperature.

### Example 1L

Oil phase I was prepared by dissolving 1.44 g hexathiol monomer and 0.36 g trimethylol propane-adduct of xylylene diisocyanate (Takenate D-110N; origin: Mitsui Chemicals, 75% polyisocyanate/25% ethyl acetate) in 18 g Perfume oil composition 2, and oil phase II was prepared by adding 0.016 g NaI in 1.3 g ethyl acetate. Oil phase II was then added into the oil phase I upon swirling. The water phase contains 0.85 g polyvinyl alcohol (PVA, Mowiol 18-88) in 60 g DI water. The oil phase and water phase were then mixed and homogenized at 8400 rpm for 2 min to form the oil-in-water emulsion. 1.3 g H₂O₂ (30 wt. % aq.) was then added to the oil-in-water emulsion. The microcapsules were obtained after overnight reaction at room temperature.

### Example 1P

0.5 g of sodium caseinate and 1.5 g of whey protein isolate (WPI) was dissolved in 65.23 g of DI water. 0.4 g CaCl₂·2 H₂O (pre-dissolved in 2 g DI water) was added to the sodium caseinate/WPI solution and stirred for 10-15 min to form the emulsifier solution. 3.0 g of trithiol monomer was added to 20 g of Perfume oil composition 1. 0.13 g of NaI was dissolved in 0.5 mL of ethanol and then added to the perfume mixture to form the oil phase. The oil phase was added to the emulsifier solution and homogenized with a T25 Ultra Turrax at 7,000 rpm for 2 min to form the emulsion. The emulsion was added to a 100 mL reactor equipped with an anchor stirrer and was stirred at 400 rpm. 1.94 g of H₂O₂ (30%) was added to the emulsion and stirred at RT for 16 hr. The reactor was heated to 45° C. and the pH was adjusted to 6.5-7. 0.8 g of transglutaminase (Activa TI, pre-dissolved in 4 g DI water) was added and stirred for 3 hr. The pH was adjusted to 5.4, and the reactor was heated to 80° C. and stirred for 30 min before being cooled to RT.

### Example 1Q

0.5 g of sodium caseinate and 1.5 g of whey protein isolate (WPI) was dissolved in 67.69 g of

DI water. 0.4 g CaCl₂·2H₂O (pre-dissolved in 2 g DI water) was added to the sodium caseinate/WPI solution and stirred for 10-15 min to form the emulsifier soln. 1.6 g of hexathiol monomer was added to 19.0 g of Perfume oil composition 2 and 1.0 g of Uvinul A Plus (UV tracer). 0.019 g of NaI was dissolved in 100 uL of ethanol and then added to the perfume mixture to form the oil phase. The oil phase was added to the emulsifier solution and homogenized with a T25 Ultra Turrax at 7,000 rpm for 2 min to form the emulsion. The emulsion was added to a 100 mL reactor equipped with an anchor stirrer and was stirred at 400 rpm. 1.39 g of H₂O₂ (30%) was added to the emulsion and stirred at room temperature for 16 hours. The reactor was heated to 45° C. and the pH was adjusted to 6.5-7. 0.8 g of transglutaminase (Activa TI, pre-dissolved in 4 g DI water) was added and stirred for 3 hr. The pH was adjusted to 5.4, and the reactor was heated to 80° C. and stirred for 30 min before being cooled to RT.

### Example 2: Morphology and Stability Analysis

Optical microscopy and scanning electron microscopy (SEM), as depicted by the figures referred to within this example, show that the polydisulfide microcapsules as prepared according to the respective processes described in example 1 exhibit spherical structures with a diameter in the micrometer range.

FIG. 1 shows an SEM image of the hexathiol-based polydisulfide microcapsule slurry prepared in example 1B.

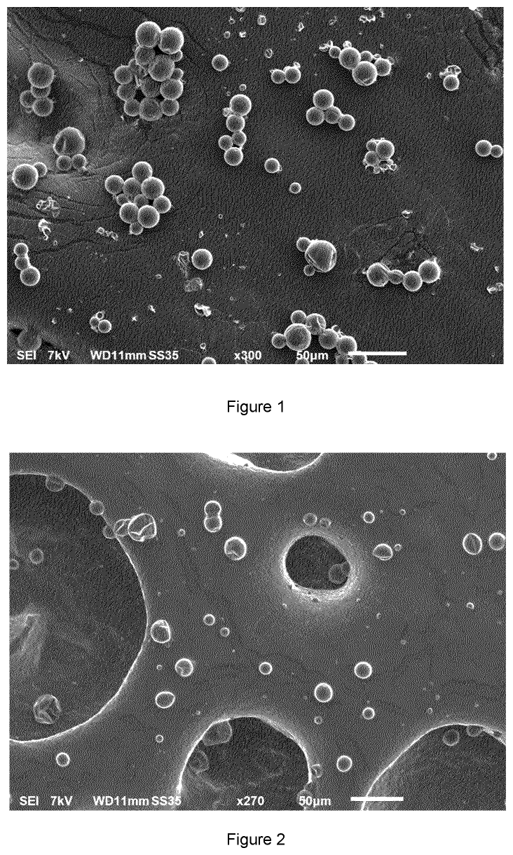

FIG. 2 shows an SEM image of the hexathiol polydisulfide microcapsule slurry prepared in example 1B 20-fold diluted by fabric softener (Stepantex VL 90A (8.8%), Calcium Chloride Sol. 10% (0.36%), Proxel GXL (0.04%), Perfume (1%), water (89.72%)) retrieved after 20 days, underlying a good stability of the microcapsules of the invention. SEM is performed under high vacuum and therefore allows to study the mechanical stability of microcapsules. Polydisulfide microcapsules which are stable under high vacuum usually exhibit good mechanical stability.

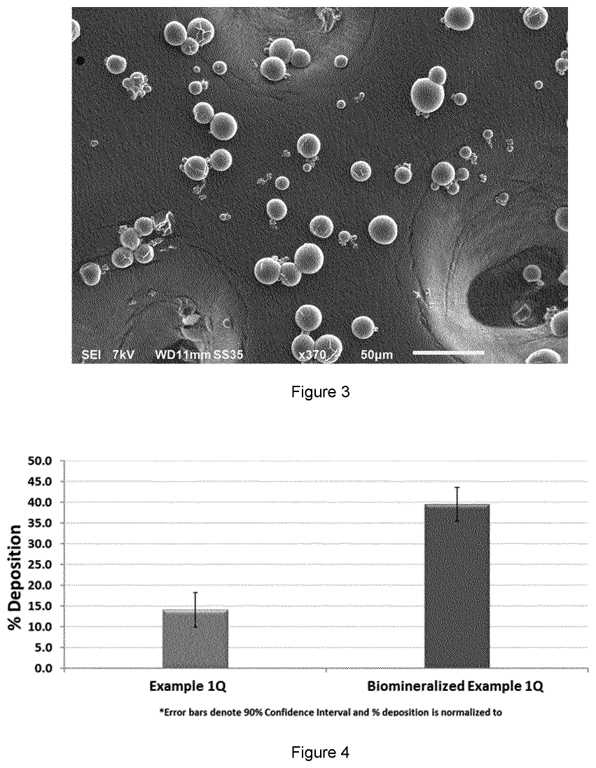

FIG. 3 shows an SEM image of the trithiol-based polydisulfide microcapsule slurry prepared in example 1I. From FIG. 3 it can be concluded that also the trithiol-based polydisulfide microcapsules are stable under high vacuum.

### Example 3: Bio-Mineralization of Example Q for the Deposition Test

The hexathiol-based disulfide microcapsules as prepared in example 1Q were coated with an additional calcium phosphate biomineral layer.

15 g of the microcapsule slurry as prepared in example 1Q is diluted in 135 g of an alkaline buffer solution at pH 9 and 4.5 mL. Subsequently 0.3 molar calcium nitrate solution is added. The mixture is stirred by anchor stirrer in a closed reactor at 250 rpm until the calcium ions have had sufficient time to interact with the anionic surface of the microcapsules.
(i) 4.5 mL of a 0.18 molar solution of dibasic sodium phosphate is added slowly with a syringe pump over 60 minutes (75 μL/min) to initiate the nucleation of mineral material at the capsule surface by precipitating the calcium cations with the phosphate anions followed by an additional 60 minutes of stirring.
(ii) Equal 7.5 mL volumes of 0.3 molar calcium nitrate and 0.18 molar sodium phosphate solutions are then simultaneously slowly added by syringe pump over 60 minutes (125 μL/min each) followed by an hour of stirring to allow for further mineral precipitation.
(iii) Equal 30 mL volumes of 0.3 molar calcium nitrate and 0.18 molar sodium phosphate solutions are then slowly added over 60 minutes (500 μL/min) simultaneously followed by 60 minutes of stirring to allow for further mineral precipitation. This process is repeated once again to generate a robust mineral shell in this example. The additions could be repeated systematically to achieve the desired mineral shell thickness and properties.

FIG. 4 shows the deposition efficiency on hair of the hexathiol-based disulfide microcapsule slurry prepared in example 1Q with and without a calcium phosphate biomineral layer. FIG. 4 shows that deposition of the microcapsules of the invention can be improved by coating a mineral layer.

### Example 4: Dynamic Headspace Analysis

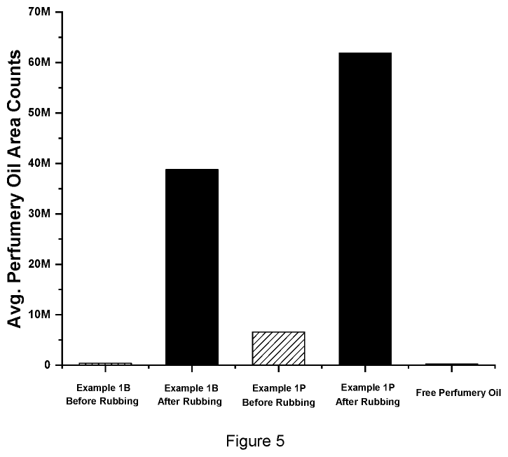

FIG. 5 shows a dynamic headspace (DHS) analysis of polydisulfide core-shell microcapsules of Example 1B before rubbing and Example 1P before rubbing diluted with deionized water in a fraction of 1:100 and dried for 24 h on a blotter paper and compares with a dynamic headspace analysis of Example 1B after rubbing and Example 1P after rubbing with friction.

The dynamic headspace (DHS) analysis shows that polydisulfide core-shell microcapsules have significantly increased area counts upon friction as compared to the same microcapsules before rubbing, proving that the polydisulfide core-shell microcapsules can protect the fragrance oil well and be ruptured by friction and have a burst release behavior of the fragrance oil.

### Example 5

### Liquid Softener Composition

A sufficient amount of exemplified microcapsules is weighed and mixed in a liquid softener composition to add the equivalent of 0.2% perfume.

TABLE 3
Liquid softener composition

| Product | Wt % |
|---|---|
| Stepantex VL 90A | 8.88 |
| Calcium Chloride Sol. 10% | 0.36 |
| Proxel GXL | 0.04 |
| Perfume | 1.00 |
| Water | 89.72 |
| TOTAL | 100 |

### Example 6

### Powder Detergent Composition

A sufficient amount of exemplified microcapsules is weighed and mixed in a powder detergent composition to add the equivalent of 0.2% perfume.

TABLE 4
Powder detergent composition

| Ingredients | Part |
|---|---|
| Anionic (Linear Alkyl Benzene Sulphonates) | 20% |
| Nonionics (Alcohol Ethoxylates (5-9 ethylene oxide) | 6% |
| Builders (zeolites, sodium carbonate) | 25% |
| Silicates | 6% |
| Sodium Sulphate | 35% |
| Others (Enzymes, Polymers, Bleach) | 7.5% |
| Spray-dried granule powder A-E | 0.5% |

### Example 7

### Liquid Detergent Composition

Microcapsules of the present invention are dispersed in a liquid detergent base described below to obtain a concentration of encapsulated perfume oil at 0.22%.

TABLE 5
Liquid detergent composition

| | Concentration |
|---|---|
| Ingredients | [wt %] |
| Sodium C14-17 Alkyl Sec Sulfonate¹⁾ | 7 |
| Fatty acids, C12-18 and C18-unsaturated²⁾ | 7.5 |
| C12/14 fatty alcohol polyglycol ether with 7 mol EO³⁾ | 17 |
| Triethanolamine | 7.5 |
| Propylene Glycol | 11 |
| Citric acid | 6.5 |
| Potassium Hydroxyde | 9.5 |
| Protease | 0.2 |
| Amylase | 0.2 |
| Mannanase | 0.2 |
| Acrylates/Steareth-20 Methacrylate structuring | 6 |
| Crosspolymer⁴⁾ | |
| Deionized Water | 27.4 |

¹⁾Hostapur ® SAS 60; Origin: Clariant
²⁾Edenor ® K 12-18; Origin: Cognis
³⁾Genapol ® LA 070; Origin: Clariant
⁴⁾Aculyn ® 88; Origin: Dow Chemical

### Example 8

### Rinse-Off Conditioner

Microcapsules of the present invention are dispersed in a rinse-off conditioner base described below to obtain a concentration of encapsulated perfume oil at 0.5%.

TABLE 6
Rinse-off conditioner composition

| | | Concentration |
|---|---|---|
| | Ingredients | [wt %] |
| A | Water deionized | 81.8 |
| | Behentrimonium Chloride ¹⁾ | 2.5 |
| | Hydroxyethylcellulose ²⁾ | 1.5 |
| B | Cetearyl Alcohol ³⁾ | 4 |
| | Glyceryl Stearate (and) PEG-100 Stearate ⁴⁾ | 2 |
| | Behentrimonium Methosulfate (and) Cetyl alcohol | 4 |
| | (and) Butylene Glycol ⁵⁾ | |
| | Ethoxy (20) Stearyl Alcohol ⁶⁾ | 1 |
| C | Amodimethicone (and) Trideceth-12 (and) | 3 |
| | Cetrimonium Chloride ⁷⁾ | |
| | Chlorhexidine Digluconate ⁸⁾ 20% aqueous solution | 0.2 |
| D | Citric acid 10% aqueous sol. till pH 3.5-4 | q.s. |
| | TOTAL: | 100 |

¹⁾ Genamin KDM P, Clariant
²⁾ Tylose H10 Y G4, Shin Etsu
³⁾ Lanette O, BASF
⁴⁾ Arlacel 165-FP-MBAL-PA-(RB), Croda
⁵⁾ Incroquat Behenyl TMS-50-MBAL-PA-(MH) HA4112, Croda
⁶⁾ SP Brij S20 MBAL-PA(RB), Croda
⁷⁾ Xiameter DC MEM-0949 Emulsion, Dow Corning
⁸⁾ Alfa Aesar

### Example 9

### Shampoo Composition

Microcapsules of the present invention are weighed and mixed in a shampoo composition to add the equivalent of 0.2% perfume.

TABLE 7
Shampoo composition

| | | Concentration |
|---|---|---|
| | Ingredients | [wt %] |
| A | Water deionized | 44.4 |
| | Polyquaternium-10 ¹⁾ | 0.3 |
| | Glycerin 85% ²⁾ | 1 |
| | DMDM Hydantoin ³⁾ | 0.2 |
| B | Sodium Laureth Sulfate ⁴⁾ | 28 |
| | Cocamidopropyl Betaine ⁵⁾ | 3.2 |
| | Disodium Cocoamphodiacetate ⁶⁾ | 4 |
| | Ethoxy (20) Stearyl Alcohol ⁶⁾ | 1 |
| C | Sodium Laureth Sulfate ⁴⁾ | 3 |
| | Glyceryl Laureate ⁷⁾ | 0.2 |
| D | Water deionized | 1 |
| | Sodium Methylparaben ⁸⁾ | 0.1 |
| E | Sodium Chloride 10% aqueous sol. | 15 |
| | Citric acid 10% aqueous sol. till pH 5.5-6 | q.s. |
| | Perfume | 0.5 |
| | TOTAL: | 100 |

¹⁾ Ucare Polymer JR-400, Noveon
²⁾ Schweizerhall
³⁾ Glydant, Lonza
⁴⁾ Texapon NSO IS, Cognis
⁵⁾ Tego Betain F 50, Evonik
⁶⁾ Amphotensid GB 2009, Zschimmer & Schwarz
⁷⁾ Monomuls 90 L-12, Gruenau
⁸⁾ Nipagin Monosodium, NIPA

### Example 10

### Antiperspirant Roll-on Emulsion Composition

Microcapsules of the present invention are weighed and mixed in antiperspirant roll-on emulsion composition to add the equivalent of 0.2% perfume.

TABLE 8
Antiperspirant roll-on emulsion composition

| | Amount |
|---|---|
| Ingredient | (wt %) |
| Steareth-2¹⁾ (Part A) | 3.25 |
| Steareth-21²⁾ (Part A) | 0.75 |
| PPG-15 Stearyl Ether³⁾ (Part A) | 4 |
| WATER deionised (Part B) | 51 |
| Aluminum Chlorohydrate 50% aqueous solution⁴⁾ (Part C) | 40 |
| Fragrance (Part D) | 1 |

¹⁾BRIJ 72; origin: ICI
²⁾BRIJ 721; origin: ICI
³⁾ARLAMOL E; origin: UNIQEMA-CRODA
⁴⁾LOCRON L; origin: CLARIAN

Part A and B are heated separately to 75° C.; Part A is added to Part B under stirring and the mixture is homogenized for 10 min. Then, the mixture is cooled under stirring; and Part C is slowly added when the mixture reached 45° C. and Part D when the mixture reached at 35° C. while stirring. Then the mixture is cooled to room temperature.

### Example 11

### Deodorant Spray Composition

Microcapsules of the present invention are weighed and mixed in antiperspirant roll-on emulsion composition to add the equivalent of 0.2% perfume.

TABLE 9
Deodorant spray composition

| Ingredient | Amount (wt %) |
|---|---|
| Ethanol 95% | 90.65 |
| Triclosan¹⁾ | 0.26 |
| Isopropyl miristate | 9.09 |

¹⁾Irgasan ® DP 300; trademark and origin: BASF

All the ingredients according to the sequence of Table 11 are mixed and dissolved. Then the aerosol cans are filled, crimp and the propellant is added (Aerosol filling: 40% active solution 60% Propane/Butane 2.5 bar).

### Example 12

### Shower-Gel Composition

Microcapsules of the present invention are weighed and mixed in the following composition to add the equivalent of 0.2% perfume.

TABLE 10
Shower gel composition

| | Amount | |
|---|---|---|
| Ingredients | (% wt) | Function |
| WATER deionized | 49.350 | Solvent |
| Tetrasodium EDTA ¹⁾ | 0.050 | Chelating agent |
| Acrylates Copolymer²⁾ | 6.000 | Thickener |
| Sodium C12-C15 Pareth Sulfate ³⁾ | 35.000 | Surfactant |
| Sodium Hydroxide 20% aqueous solution | 1.000 | pH adjuster |
| Cocamidopropyl Betaine⁴⁾ | 8.000 | Surfactant |
| Methylchloroisothiazolinone and | 0.100 | Preservative |
| Methylisothiazolinone⁵⁾ | | |
| Citric Acid (40%) | 0.500 | pH adjuster |

¹⁾ EDETA B POWDER; trademark and origin: BASF
²⁾CARBOPOL AQUA SF-1 POLYMER; trademark and origin: NOVEON
³⁾ ZETESOL AO 328 U; trademark and origin: ZSCHIMMER & SCHWARZ
⁴⁾TEGO-BETAIN F 50; trademark and origin: GOLDSCHMIDT
⁵⁾KATHON CG; trademark and origin: ROHM & HASS

### Example 13

### Unit Dose Formulation

A sufficient amount of exemplified microcapsules is weighed and mixed in a unit dose formulation to add the equivalent of 0.2% perfume.

The unit dose formulation can be contained in a PVOH (polyvinyl alcohol) film.

TABLE 11
Unit dose composition

| | Concentration |
|---|---|
| Ingredients | [wt %] |
| C12-C14 alkyl poly ethoxylate | 15 |
| C12-C14 alkyl poly ethoxylate sulfate Mono Ethanol | 9.5 |
| Amine salt | |
| Linear Alkylbenzene sulfonic acid | 17 |
| Citric Acid | 0.5 |
| C12-C18 Fatty Acid | 17 |
| Enzymes | 1.2 |
| Fluorescent brightener | 0.3 |
| 1,2 propanediol | 12 |
| Glycerol | 9 |
| Sodium Hydroxide | 1 |
| Mono Ethanol Amine | 6 |
| PDMS | 2.5 |
| Potassium sulphite | 0.2 |
| water | 8.8 |
| Total | 100 |

### Example 14

### Cosmetic Day Cream

A sufficient amount of exemplified microcapsules is weighed and mixed into a cosmetic skin cream (see composition below) at a concentration of 5% w/w.

TABLE 12
Cream composition

| | | Amounts |
|---|---|---|
| Phase | Ingredients | (%) |
| A | Ethoxylated Fatty Alcohol Ester ¹⁾ | 5 |
| | Cetyl alcohol | 0.5 |
| | Ceteth-20 (and) Glyceryl Stearate (and) PEG-6 | 4 |
| | Stearate (and) Steareth-20 ²⁾ | |
| | Squalan ³⁾ | 1 |
| | Paraffin oil | 2 |
| | Petrolatum | 6 |
| B | Water | 55.85% |
| | Hydrated coacervate phase | 20 |
| C | Propylene glycol | 5 |
| | DMDM Hydantoin (and) Iodopropynyl | 0.15 |
| | Butylcarbamate 4) | |
| D | Sodium Carbomer | 0.2 |
| E | Perfume | 0.3 |

¹⁾ ARLATONE 985
²⁾ TEFOSE 2561
³⁾ COSBIOL
4) GLYDANT PLUS.

1. A process for the preparation of a polydisulfide core-shell microcapsule slurry, comprising the steps of:
a) dissolving at least one polythiol monomer in a hydrophobic material to form an oil phase; and
b) dispersing the oil phase obtained in step a) into an aqueous phase to form an oil-in-water emulsion; and
c) applying sufficient conditions to induce thiol oxidation at the oil-water interface to form a polydisulfide core-shell microcapsule slurry,
wherein an oxidation catalyst is added in the oil phase in step a), and/or an oxidation catalyst is added in the oil-in-water emulsion obtained after step b),
wherein a stabilizer is added in the oil phase in step a) and/or in the aqueous phase in step b), and
wherein an oxidant is added in the aqueous phase in step b) and/or in the oil-in-water emulsion obtained after step b).

2. The process according to claim 1, wherein it comprises the steps of:
a) dissolving at least one polythiol monomer in a hydrophobic material, to form an oil phase;
b) dispersing the oil phase obtained in step a) into an aqueous phase to form an oil-in-water emulsion; and
c) adding an oxidant in the oil-in-water emulsion under stirring to induce thiol oxidation at the oil-water interface to form a polydisulfide core-shell microcapsule slurry,
wherein an oxidation catalyst is added in the oil phase in step a), and/or an oxidation catalyst is added in the oil-in-water emulsion obtained after step b), and
wherein a stabilizer is added in the oil phase in step a) and/or in the aqueous phase in step b).

3. The process according to claim 1, wherein the oxidation catalyst is added in the oil phase in step a).

4. The process according to claim 1, wherein the polythiol monomer is selected from the group consisting of difunctional thiol monomers, trifunctional thiol monomers, tetrafunctional thiol monomers, pentafunctional thiol monomers, hexafunctional thiol monomers, heptafunctional thiol monomers, octafunctional thiol monomers, thiolated polysaccharides, thiolated chitosan, thiolated proteins, thiolated pectin, thiolated polyols, hydrolysed/reduced keratin, proteins with two or more thiol groups and mixtures thereof.

5. The process according to claim 1, wherein the polythiol monomer is selected from the group consisting of difunctional thiol monomers, 1,2-ethanedithiol, 1,3-propanedithiol, 1,4-butanedithiol, 1,5-pentanedithiol, 1,6-hexanedithiol, 1,8-octanedithiol, 1,9-nonanedithiol dithioerythritol, 1,4-butanediol bis(thioglycolate), 4,4′-biphenyldithiol, ethylene bis(thioglycolate), ethylene glycol bis(3-mercaptopropionate), 1,10-decanedithiol, 1,11-undecanedithiol, 1,16-hexadecanedithiol, 1,4-benzenedithiol, 3,7-dithia-1,9-nonanedithiol, 4,4′-thiobisbenzenethiol, hexa(ethylene glycol)dithiol, bis(2-mercaptoethyl) sulfide, 3,6-dioxa-1,8-octanedithiol, 1,5-dimercaptonaphthalene, 1,3-benzenedithiol, 2,3-butanedithiol, dithiothreitol, 2,3-dimercapto-1-propanol 2,2′-(ethylenedioxy)diethanethiol, hexa(ethylene glycol) dithiol, tetra(ethylene glycol) dithiol, polyethylene glycol dithiol, trifunctional thiol monomers, tris[2-(3-mercaptopropionyloxy)ethyl]isocyanurate, hexafunctional thiol monomers, dipentaerythritol hexakis(3-mercaptopropionate), and mixtures thereof.

6. The process according to claim 1, wherein the stabilizer is a colloidal stabilizer.

7. The process according to claim 1, wherein the oxidant is iodine, iodide salt, iron (II) sulfate, hydrogen peroxide, iodobenzene diacetate, ammonium persulfate, potassium ferricyanide, ammonium perborate, ferric chloride, potassium dichromate, potassium bromide, bromine, triiodide salt, copper (II) sulfate, N-bromosuccinimide, or mixtures thereof, preferably hydrogen peroxide.

8. The process according to claim 1, wherein the oxidant is used in a molar ratio of thiol functional groups to oxidant of 1:10 to 1:0.01.

9. The process according to claim 1, wherein the oxidation catalyst is iodine, iodobenzene diacetate, a bromide salt, an iodide salt, or a mixture thereof.

10. The process according to claim 1, wherein at least one further polyfunctional monomer is added to the process.

11. The process according to claim 1, comprising an additional drying step d) wherein the polydisulfide core-shell microcapsule slurry is dried to form a polydisulfide core-shell microcapsule powder.

12. A polydisulfide core-shell microcapsules as prepared by the process according to claim 1, comprising
a) an oil-based core comprising a hydrophobic material; and
b) a polymeric shell comprising disulfide bonds formed from the oxidation from at least one polythiol monomer with an oxidant in the presence of an oxidation catalyst.

13. A perfuming composition comprising
a) polydisulfide core-shell microcapsules as defined in claim 12;
b) at least one ingredient selected from the group consisting of a perfumery carrier and a perfumery base,
c) optionally at least one active ingredient;
d) optionally at least one perfuming co-ingredient; and
e) optionally at least one perfumery adjuvant.

14. A consumer product comprising
a) a personal care active base or a fabric care active base; and
b) polydisulfide core-shell microcapsules as defined in claim 12.

15. The consumer product according to claim 14, wherein the consumer product is in the form of a personal care composition, home care composition or fabric care composition.

16. The process for the preparation of a polydisulfide core-shell microcapsule slurry according to claim 1, wherein the hydrophobic material is a perfume oil.

17. The process for the preparation of a polydisulfide core-shell microcapsule slurry according to claim 1, wherein the polythiol monomer is ethylene glycol bis(3-mercaptopropionate).

18. The process for the preparation of a polydisulfide core-shell microcapsule slurry according to claim 1, wherein the stabilizer is a colloidal stabilizer selected from the group consisting of polyvinyl alcohol, whey protein isolate, sodium caseinate, gum arabic, sugar beet pectin, gelatin, a protein, soy protein, potato protein, hydrolyzed silk protein, and combinations thereof.

19. The process for the preperation of a polydisulfide core-shell microcapsule slurry according to claim 1, wherein the oxidant is hydrogen peroxide.

20. The process for the preperation of a polydisulfide core-shell microcapsule slurry according to claim 1, wherein the oxidant is used in a molar ratio of thiol functional groups to oxidant of 1:2 to 1:1.
